(19) United States
(12) Patent Application Publication
(10) Pub. No.: US 2019/0008992 A1
(43) Pub. Date: Jan. 10, 2019

(54) Fragrance Discharge Device

(71) Applicant: Pium Labs, Inc., Fort Lee, NJ (US)

(72) Inventors: Jaeyeon Kihm, Fort Lee, NJ (US); Sung Jae Hwang, Seongnam-si (KR); Jung Ho Kim, Seoul (KR)

(21) Appl. No.: 16/121,299

(22) Filed: Sep. 4, 2018

Related U.S. Application Data
(63) Continuation-in-part of application No. PCT/KR2017/002277, filed on Feb. 3, 2017.

(30) Foreign Application Priority Data
Mar. 4, 2016 (KR) 10-2016-0026674
Jun. 13, 2016 (KR) 10-2016-0073072

(51) Int. Cl. A61L 9/14; A61L 9/03

(52) U.S. Cl. CPC A61L 9/14; B05B 12/02; A61L 9/03

(57) ABSTRACT

Provided is a fragrance discharge device. The fragrance discharge device includes a storage configured to store a fragrance material; a body to which the storage is mounted; a discharge portion configured to discharge, to an outside of the body, at least a portion of the fragrance material supplied from the storage; and a controller configured to control the discharge portion to discharge the fragrance material based on context information including at least one of user information and environmental information around the body.

This application is related to concurrently filed U.S. application Ser. No. ______, entitled “Fragrance Discharge Device and Control Method Thereof,” and concurrently filed U.S. application Ser. No. ______, entitled “Mobile Fragrance Discharge Device,” which are herein incorporated by reference in their entirety for all purposes.

### TECHNICAL FIELD

Embodiments relate to a fragrance discharge device.

### BACKGROUND

A fragrance refers to a pleasant odor that may come from flowers, incense, perfume, etc. It has long been known effect that a specific fragrance may affect psychology and emotions of a user. Therefore, people are using perfume for various purposes, such as mood stabilization, psychological stability, and ventilation.

Perfume is generally provided in a liquid form and may be stored and used in a container provided with a device capable of dispensing liquid. In general, perfume in a container is kept at a desired place and used there or by being dispensed by the user over a body part such as a wrist and a neck of the user or clothes of the user as desired.

However, perfume according to the related art is placed in a specific space to spread a fragrance within the space through a natural diffusion method or to discharge a fragrance material at desired intervals.

Such perfume according to the related art may not actively operate. For example, such perfume may not apply spatial characteristics of a specific space, for example, an office, a private room, a kitchen, and a toilet, and, in addition to the spatial characteristics, may not apply a condition of a user.

Accordingly, perfume may be discharged unnecessarily although the user is absent in the specific space, may not be discharged at a proper point in time at which the perfume needs to be discharged, or may be discharged periodically in a situation in which a plurality of persons is present and discharging of a specific fragrance is improper. That is, perfume according to the related art may not suitably meet the potential intent and needs of the user.

### SUMMARY

Embodiments provide a fragrance discharge device that may operate based on context information of a user.

Embodiments also provide a fragrance discharge device that may discharge a fragrance material although a user does not directly manipulate a device.

Embodiments also provide a fragrance discharge device that may control discharging of a fragrance material in conjunction with an external device.

According to an aspect, there is provided a fragrance discharge device including a storage configured to store a fragrance material; a body to which one or more storages are mounted; a discharge portion provided to the body and configured to discharge, to an outside, the fragrance material supplied from the storage; and a controller configured to recognize a presence or an absence of a target present around the body and to control the discharge portion to discharge the fragrance material or to control a discharge direction of the fragrance material based on the presence or the absence of the target and a location of the target.

Also, the controller may be configured to control discharging of the fragrance material based on the presence or the absence of the target, the location of the target, and attribute information of the target, and the attribute information of the target may include at least one of a size and a type of the target.

Also, the fragrance discharge device may further include a sensor portion provided to the body and configured to detect the target present around the body.

Also, the sensor portion may include at least one of a location recognition sensor, a proximity sensor, a distance sensor, and an image sensor.

Also, the controller may be configured to adjust at least one of a type, an amount, a discharge direction, and a discharge pattern of the fragrance material that is discharged through the discharge portion.

Also, the fragrance discharge device may further include a communicator configured to communicably connect to an external device to control discharging of the fragrance material in conjunction with the external device.

Also, the controller may be configured to control discharging of the fragrance material based on the presence or the absence of the target, the location of the target, and attribute information of the target, and the target present around the body and the attribute information of the target may be received from the external device.

Also, the external device may be a user device, and a user interface for providing a list of external devices interacting with the fragrance discharge device or controlling the fragrance discharge device may be provided to the user device.

Also, the controller may be configured to control discharging of the fragrance material based on the presence or the absence of the target, the location of the target, and attribute information of the target, the external device may be a device capable of discharging the fragrance material, and the controller may be configured to control discharging of the fragrance material in conjunction with the external device based on the attribute information of the target.

Also, the external device may be a device capable of discharging the fragrance material, and the controller may be configured to control discharging of the fragrance material in conjunction with the external device based on a relative location between the external device and a user.

Also, the external device may be a multimedia player, and the controller may be configured to control discharging of the fragrance material based on content information of multimedia being played on the external device.

Also, the controller may be configured to control discharging of the fragrance material based on modified attribute information of the target in response to the attribute information of the target being modified.

Also, the controller may be configured to control the discharge portion based on user information of the fragrance discharge device with the presence or the absence of the target and the attribute information of the target.

Also, the user information may include at least one of a relative location between the fragrance discharge device and the user, body information of the user, and preference information of the user on a specific fragrance material.

Also, the fragrance discharge device may further include an artificial intelligence (AI) learner configured to learn a control history of the controller on discharging of the fragrance material based on at least one of the presence or the absence of the target, the location of the target, and the attribute information of the target, and the user information, and to provide a result of the learning to the controller. The controller may be configured to control the discharge portion using a scheme recommended by the AI learner.

Also, the AI learner may be configured to learn the control history using a time series data analysis.

Also, the body may include one or more mounting holes into which the one or more storages are inserted, respectively, and the storage may be replaceably mounted to the mounting hole.

Also, a nozzle through which the fragrance material is discharged may be mounted to be rotatable relative to the body to change a discharge direction of the fragrance material.

According to an aspect, there is provided a fragrance discharge device including a storage configured to store a fragrance material; a discharge portion configured to connect to the storage and to discharge, to an outside, the fragrance material supplied from the storage; a body to which one or more storages are mounted; a sensor portion configured to sense a presence and a location of a target present around the body; a communicator configured to communicably connect to an external device; and a controller configured to control the discharge portion to discharge the fragrance material or to control a discharge direction of the fragrance material based on the presence or an absence of the target and the location of the target transferred from the sensor portion. The controller is configured to transmit at least one of a stored amount of the fragrance material and discharge information of the fragrance material to the external device through the communicator and to control the discharge portion based on a control signal received from the external device through the communicator.

Also, the controller may be configured to control discharging of the fragrance material based on the presence or the absence of the target, the location of the target, and attribute information of the target, and at least a portion of the attribute information of the target may be received from the external device.

According to an aspect, there is provided a fragrance discharge device to communicably connect to an external device capable of discharging one or more fragrance materials, the fragrance discharge device including a storage configured to store a fragrance material; a body to which one or more storages are mounted; a discharge portion provided to the body and configured to discharge, to an outside, the fragrance material supplied from the storage; a sensor portion configured to sense a presence and a location of a target present around the body; a communicator configured to communicably connect to the external device; and a controller configured to control the discharge portion to discharge the fragrance material or to control a discharge direction of the fragrance material based on the presence or an absence of the target and the location of the target transferred from the sensor portion. The controller is configured to control fragrance material discharge of the external device through the communicator.

Also, the controller may be configured to control the discharge portion to control discharging of the fragrance material based on environmental information around the body.

A fragrance discharge device according to embodiments may control discharging of a fragrance material based on context information including user information or environmental information.

Also, the fragrance discharge device may discharge the fragrance material although a user does not directly manipulate the device.

Also, the fragrance discharge device may control discharging of the fragrance material within a space in conjunction with an external device.

Also, the fragrance discharge device may economically use the fragrance material by controlling discharging of the fragrance material based on the context information.

### BRIEF DESCRIPTION OF DRAWINGS

FIG. 1 is a block diagram illustrating a configuration of a fragrance discharge device according to an embodiment.

FIG. 2 is a perspective view of the fragrance discharge device including the configuration of FIG. 1.

FIG. 3 is a perspective view of a fragrance discharge device according to another embodiment.

FIG. 4 is a block diagram illustrating a configuration of the fragrance discharge device of FIG. 1 and an external device interacting therewith.

FIGS. 5A through 5D illustrate examples of a user interface of an external device according to an embodiment.

FIG. 6 is a block diagram illustrating a configuration of the fragrance discharge device of FIG. 1 and another external device interacting therewith.

FIG. 7 is a flowchart illustrating a control method of a fragrance discharge device according to an embodiment.

FIG. 8 is a flowchart illustrating a control method of a fragrance discharge device according to another embodiment.

FIG. 9 is a flowchart illustrating a control method of a fragrance discharge device according to another embodiment.

FIG. 10 is a flowchart illustrating a control method of a fragrance discharge device according to another embodiment.

FIG. 11 is a flowchart illustrating a control method of a fragrance discharge device according to another embodiment.

FIGS. 12A and 12B illustrate examples in which a fragrance discharge device interacts with a multimedia player according to an embodiment.

FIG. 13 illustrates an example in which a fragrance discharge device interacts with another multimedia player according to an embodiment.

FIG. 14 illustrates an example in which a fragrance discharge device interacts with an external device according to an embodiment.

FIG. 15 illustrates an example in which a fragrance discharge device interacts with various external devices according to an embodiment.

FIG. 16 illustrates another example in which a fragrance discharge device interacts with various external devices according to an embodiment.

FIG. 17 illustrates an example in which a plurality of fragrance discharge devices operates through mutual interaction according to an embodiment.

FIG. 18 illustrates another example in which a plurality of fragrance discharge devices operates through mutual interaction according to an embodiment.

FIG. 19 illustrates another example in which a plurality of fragrance discharge devices operates through mutual interaction according to an embodiment.

FIG. 20 illustrates another example in which a plurality of fragrance discharge devices operates through mutual interaction according to an embodiment.

FIG. 21 illustrates an example in which a fragrance discharge device operates based on a user schedule according to an embodiment.

FIG. 22 is a block diagram illustrating a configuration of a fragrance discharge device according to an embodiment.

FIG. 23 illustrates a learning process of an artificial intelligence (AI) learner of FIG. 22.

### DETAILED DESCRIPTION

Hereinafter, embodiments will be described in detail with reference to the accompanying drawings such that those having ordinary skill in the art to which the present disclosure pertains may easily practice the present disclosure. The present disclosure may, however, be embodied in many different forms and should not be construed as being limited to the embodiments set forth herein. In addition, in the description of the present disclosure, detailed description of well-known related structures or functions will be omitted when it is deemed that such description will cause ambiguous interpretation of the present disclosure.

The terminology used herein is for the purpose of describing particular embodiments only and is not intended to be limiting. As used herein, the singular forms “a,” “an,” and “the,” are intended to include the plural forms as well, unless the context clearly indicates otherwise.

It will further be understood that the terms “comprises,” “comprising,” “includes,” and/or “including,” when used herein, specify the presence of stated features, integers, steps, operations, components, and/or parts, but do not preclude the presence or addition of one or more other features, integers, steps, operations, components, parts, and/or groups thereof.

Further, the following embodiments are provided for further clear explanation for those of ordinary skill in the art and thus, shapes and sizes of elements shown in the drawings may be exaggerated for the clear explanation.

Configuration of Fragrance Discharge Device

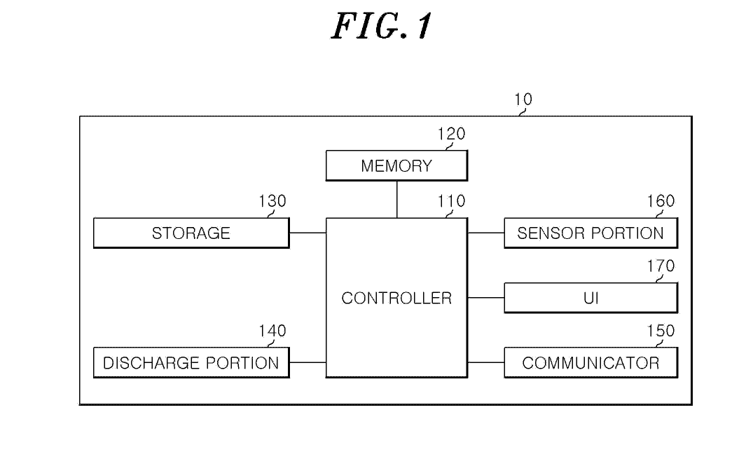

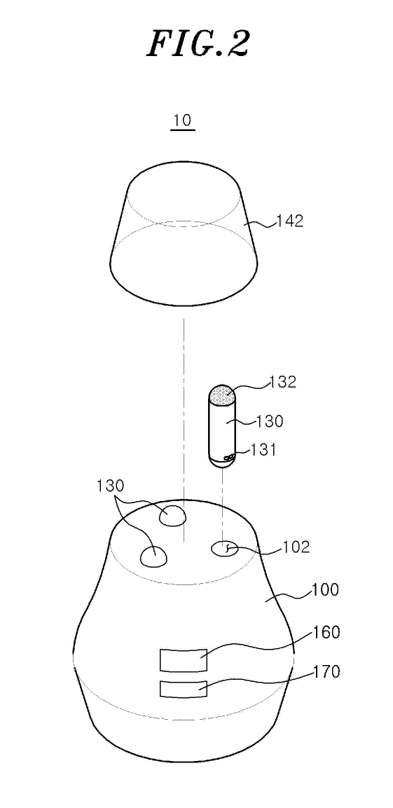

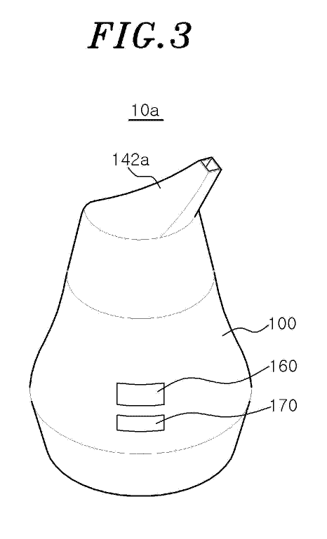

FIG. 1 is a block diagram illustrating a configuration of a fragrance discharge device according to an embodiment and FIG. 2 is a perspective view of the fragrance discharge device including the configuration of FIG. 1. FIG. 3 is a perspective view of a fragrance discharge device according to another embodiment.

Referring to FIGS. 1 through 3, a fragrance discharge device 10 according to an embodiment is configured to discharge at least one fragrance material stored inside to an outside.

In detail, the fragrance discharge device 10 may include a storage 130 configured to store a fragrance material, a body 100 to which one or more storages 130 are mounted, a discharge portion 140 provided to the body 100 and configured to discharge, to an outside, the fragrance material supplied from the storage 130, a controller 110 configured to control the discharge portion 140 to discharge, to the outside, the fragrance material stored in the storage 130, and a memory 120 configured to store data.

Here, the fragrance material refers to a material capable of emitting a fragrance recognizable through an olfactory sense of a person and is not limited to a state of a material stored in the storage 130 or a state of the material discharged through the discharge portion 140. That is, the fragrance material may be stored in at least one of a liquid state, a gas state, and a solid state in the storage 130 of the fragrance discharge device 10. Also, the fragrance used herein may also represent a scent and a smell, etc., and thus may be interchangeably used therewith. For example, the fragrance material may be stored in the liquid state in the storage 130 and may be evaporated and then discharged to the outside of the body 100.

Also, the fragrance material may have one or more fragrances. For example, if the fragrance material has a plurality of fragrances, the fragrance material may be a mixture of a plurality of materials each capable of emitting a fragrance.

The body 100 is accommodable within a predetermined space and has a size and a shape capable of accommodating one or more storages 130, the controller 110, and the like. The body 100 may be formed using a plastic or metal material, and may have a streamlined appearance in a pot shape as illustrated in the figures, and may be in an approximately rectangular box shape. However, it is provided as an example only and the overall shape of the body 100 may be modified if necessary. In the meantime, the body 100 may be disposed in a specific space and used only at a corresponding location, and may be mounted on a moving object such as a vehicle. A lower portion of the body 100 may be in a flat shape to be safely placed within the specific space. Also, the body 100 may include a mounting hole 102 into which each of the one or more storages 130 may insert. An upper cover 142 may be provided on a top of the body 100 to protect the storage 130 and to achieve an aesthetic appearance. The upper cover 142 may have a circular cross-section based on a shape of the top of the body 100, and, without being limited thereto, may have a cross-section in various shapes based on the shape of the body 100. Also, a top of the upper cover 142 may be open to discharge, to the outside, the fragrance material supplied from the storage 130. Also, an entire top surface of the upper cover 142 may be sealed and may include a plurality of discharge holes through which the fragrance material is discharged to the outside.

The storage 130 is configured to store a fragrance material in a predetermined state. Herein, the present embodiment is described based on an example in which the fragrance material is stored in a liquid state and the storage 130 is in an elongated-capsule shape to store the fragrance material. However, as described above, the fragrance material may be stored in various states and the storage 130 may be in a shape corresponding thereto. For example, the fragrance material may be provided in a powder form that includes solid particles. In this case, the storage 130 may include a structure, for example, a membrane to which powders may be attached and fastened. Herein, three storages 130 may be provided each in a cartridge form, and may store different fragrance materials, respectively. Each storage 130 may be inserted into each corresponding mounting hole 102 formed in the body 100. Accordingly, once the fragrance material stored in the storage 130 is all used and needs to be replaced, a user may simply replace the corresponding storage 130. The storage 130 may be formed using a material, such as glass and plastic, and may be molded using an anticorrosive material against chemical components of various fragrance materials. Also, the storage 130 may be formed using a transparent material. In this case, although the storage 130 is mounted to the body 100, a remaining amount of the fragrance material may be easily verified through a transparent window (not shown) provided to the body 100. One or more discharge holes 132 each with a minute size may be formed in an upper portion of the storage 130. Accordingly, the fragrance material stored in the storage 130 may be evaporated through the discharge portion 140 and discharged to the outside of the storage 130 through the discharge hole 132. Further, a device capable of controlling an environmental factor, such as a temperature and a humidity, may be provided in the body 100 such that the fragrance material stored in the storage 130 may not be changed and may be stably stored. For example, if an optimal temperature is required to enhance effect of emitting the fragrance material, a temperature maintaining device, such as a thermoelectric element provided in the body 100, may maintain the storage 130 to be in the optimal temperature.

The discharge portion 140 is provided to discharge, to the outside, the fragrance material stored in the storage 130. Here, discharging of the fragrance material may include dispensing the fragrance material to the outside of the fragrance discharge device 10 at predetermined pressure by directly applying a physical external force to the fragrance material and naturally emitting the fragrance material without applying the direct external force. For example, spraying the fragrance material using a predetermined device and naturally evaporating the fragrance material may be understood as discharging by the discharge portion 140. Various fragrance materials each stored in the corresponding storage 130 may be discharged to the outside using, for example, an ultrasonic vibration scheme, a natural evaporation scheme, a heating scheme, and a spray scheme. For example, an ultrasonic vibrator (not shown) may be provided in each storage 130 and an electrode portion 131 may be provided on each storage 130. Accordingly, once the storage 130 inserts into the mounting hole 102 of the body 100, an electrode provided to the body 100 and the electrode portion 131 of the storage 130 may be connected to each other to electrically control the ultrasonic vibrator. Although not illustrated herein, when the storage 130 inserts into the mounting hole 102 of the body 100 and then the upper cover 142 is provided to the body 100, an electrode provided to the upper cover 142 may be connected to an electrode provided on an upper side of the storage 130 to electrically control the ultrasonic vibrator.

Referring to FIG. 3, in a fragrance discharge device 10a according to another embodiment, an upper cover 142a with a nozzle may be rotatably mounted to the body 100. Accordingly, a discharge direction of a fragrance material may be controlled through rotation of the upper cover 142a. In an embodiment, an actuator (not shown) configured to rotate the upper cover 142a may be provided in the body 100 such that the upper cover 142a may automatically rotate under control of the controller 110. When the storage 130 needs to be replaced due to a lack of an amount of the fragrance material remaining in the storage 130, the controller 110 may control the discharge portion 140 to temporarily suspend discharging of the fragrance material.

The controller 110 may be provided in the body 100 and may be any suitable processing device capable of receiving and transmitting digital electronic signals such as, by way of example, a microprocessor, a microcontroller, a digital integrated circuit, etc. Controller 110 may further include or be in communication with a memory 120. The controller 110 may control the discharge portion 140 based on context information including at least one of user information of a user using the fragrance discharge device 10 and environmental information around the body 100 such that the fragrance material stored in the storage 130 may be discharged to the outside. The controller 110 may control at least one of a type, an amount, a discharge direction, and a discharge pattern of the fragrance material that is discharged through the discharge portion 140. Here, the user information refers to information associated with a body or a concept of the user and may include at least one of a location of the user within a space in which the fragrance discharge device 10 is installed, a relative location between the user and the fragrance discharge device 10, a type of a target, such as a person and an animal, toward which the fragrance material is discharged, an olfactory sensitivity of the user, a preference of the user on a specific fragrance material, and body information such as an emotion, stress, and a concept of the user, a change in an action of the user, and a motion of the user. Here, the body information of the user may be measured through a sensor portion 160 or a sensor portion 260 provided to an external device 20, which is described below. Also, information on the emotion, the stress, or the concept of the user may be verified based on the measured body information. Also, the environmental information may refer to information on an environment around a space in which the fragrance discharge device 10 is present, and may include at least one of characteristics of the space in which the fragrance discharge device 10 is present, for example, an ideal concept of space including a virtual space and a physical space such as a room, an office, a closet, a vehicle, and an airplane, illuminance, odor information, ventilation information, time information, and weather information. Further description related to various embodiments in which the fragrance discharge device 10 operates based on the context information is made below.

The memory 120 may store a variety of information sensed at the sensor portion 160, information exchanged with the external device 20 through a communicator 150, data input from the user and data provided to the user through a user interface (UI) 170, and the like. For example, the memory 120 may store user information including motion information of the user and biometric information of the user, the environmental information, state information of the fragrance discharge device 10, and discharge information of the fragrance material.

The fragrance discharge device 10 according to an embodiment may further include the UI 170 provided to the body 100. The UI 170 may be provided in a form of a touch display device or a light emitting diode (LED) display on the outer surface of the body 100. Thus, if a remaining amount of the fragrance material is insufficient, the fragrance discharge device 10 may provide an alert notification to the user through the UI 170. Also, if the remaining amount of the fragrance material is insufficient, the fragrance discharge device 10 may inform the user of the insufficiency through an alert sound. Also, the fragrance discharge device 10 may directly receive an input from the user through a touch display device to operate in a predetermined mode. For example, the user may select a desired fragrance material through the UI 170. Also, the UI 170 may display a type of a mode currently in operation, information on the fragrance material, and a scheduled replacement time. When a plurality of storages 130 is provided, the UI 170 may display a remaining amount of a fragrance material of each storage 130 and may also display a remaining amount acquired by adding up amounts of fragrance materials remaining in the respective storages 130. If the fragrance discharge device 10 malfunctions, the UI 170 may di splay corresponding error information.

The fragrance discharge device 10 according to an embodiment may further include the sensor portion 160. The sensor portion 160 is configured to actively detect environmental information around the fragrance discharge device 10 and may include at least one of an odor sensor, a proximity sensor, an illuminance sensor, a temperature sensor, a sound sensor, an infrared (IR) sensor, and an image sensor. The sensor portion 160 may transmit sensing information to the controller 110 and the controller 110 may control discharging of the fragrance material by controlling the discharge portion 140 based on the received information. For example, the sensor portion 160 may be the image sensor or the IR sensor and may recognize a target present around the fragrance discharge device 10 and transmit corresponding information to the controller 110. A target may be any object or environment to which a fragrance may be applied such as, by way of example, a person, animal, body part, room, bathroom, toilet, car, etc. The controller 110 may control the discharge portion 140 based on attribute information of the target. Accordingly, when a person or an object is present within a predetermined distance from the fragrance discharge device 10, the controller 110 may control the discharge portion 140 to temporarily suspend discharging of the fragrance material or to adjust a discharge direction such that the fragrance material may not be directly discharged toward the person or the object. Also, the controller 110 may adjust a discharge distance, intensity, or a period of the fragrance material such that the discharged fragrance material may not be directly transferred to the person or the object.

The fragrance discharge device 10 according to an embodiment may further include the communicator 150. The communicator 150 is provided such that the fragrance discharge device 10 may control discharging of a fragrance material in conjunction with a smartphone, a multimedia player, or another external device capable of discharging the fragrance material. For example, the fragrance discharge device 10 may connect to a mobile device, such as a smartphone of the user, through the communicator 150 and the user may control fragrance material discharge of the fragrance discharge device 10 by controlling the controller 110. Also, the fragrance discharge device 10 may recognize user information and environmental information around the space in which the fragrance discharge device 10 is installed using various sensors mounted to the mobile device and may transmit the recognized information to the controller 110 through the communicator 150. The fragrance discharge device 10 may connect to the external device 20 in a wired or wireless manner and may communicate therewith using one of, for example, a universal serial bus (USB), Infrared Data Association (IrDA), wireless fidelity (WiFi), ultra-wideband (UWB), Bluetooth, ZigBee, code division multiple access (CDMA), wideband code division multiple access (WCDMA), Long Term Evolution (LTE), and LTE-Advanced (LTE-A). That is, the fragrance discharge device 10 may connect to the external device 20 in a wired manner using a USB, may directly connect thereto in a wireless manner through WiFi, and may also connect thereto in a wireless manner through an access point (AP).

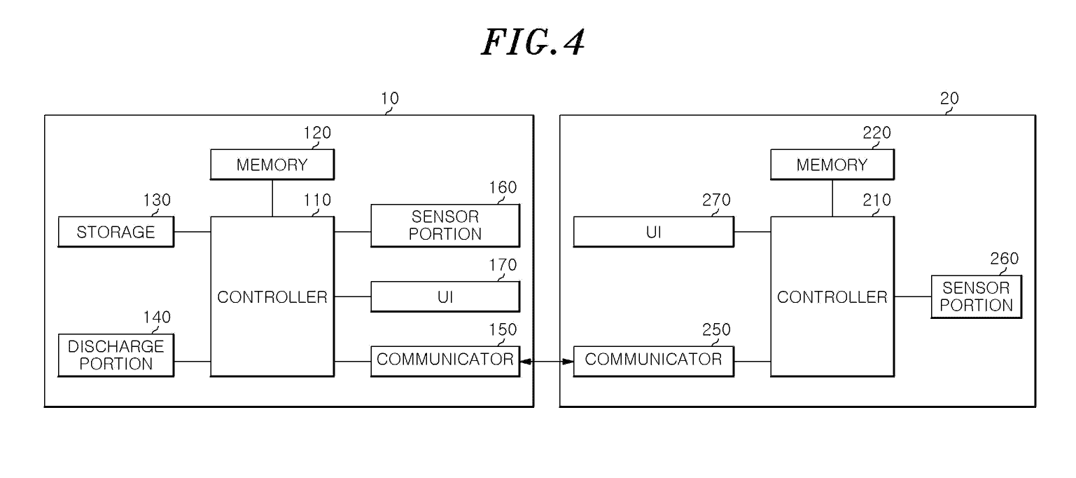

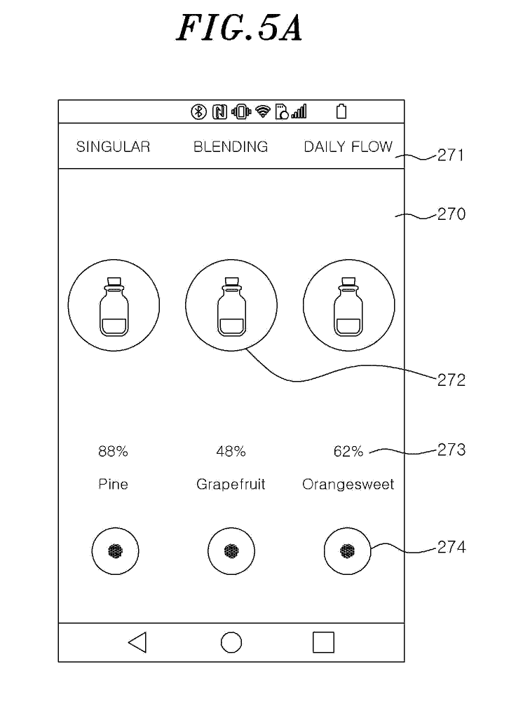

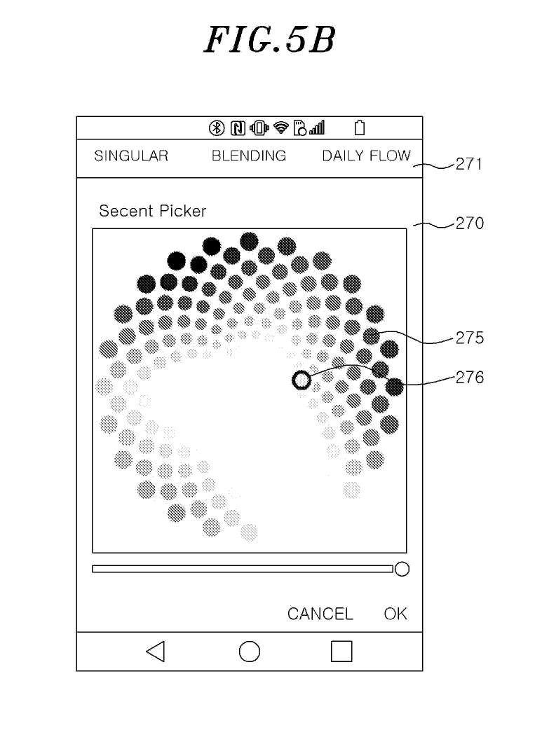

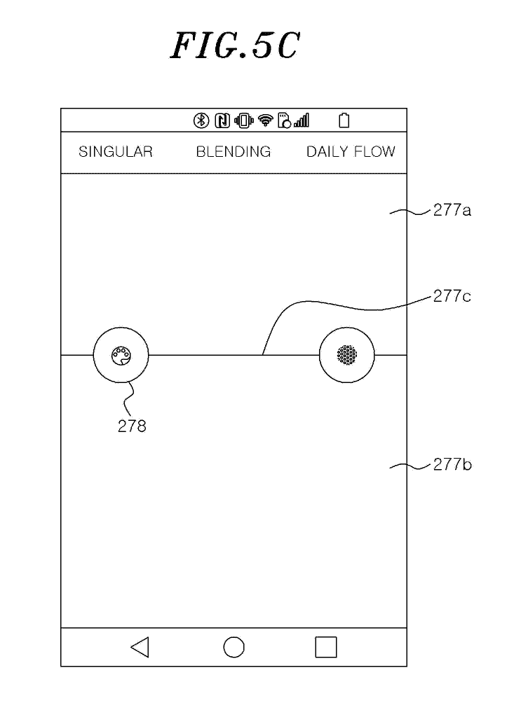

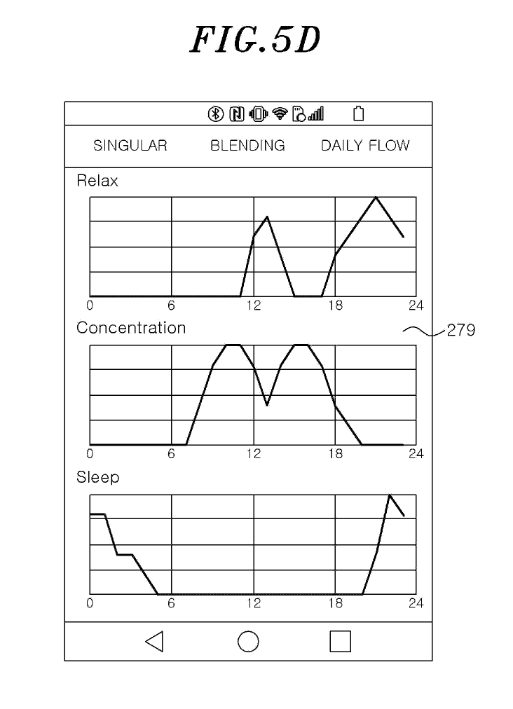

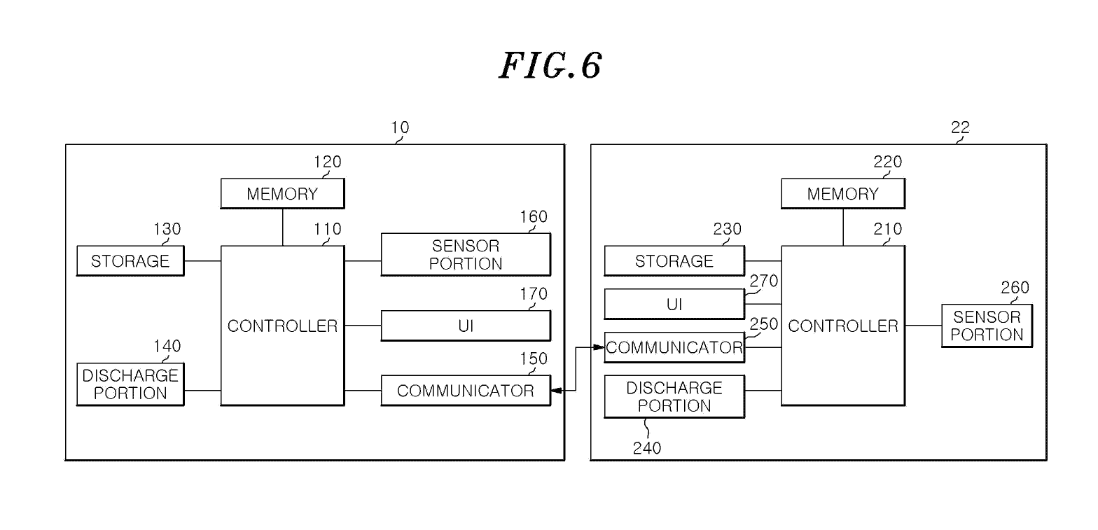

FIG. 4 is a block diagram illustrating a configuration of the fragrance discharge device of FIG. 1 and an external device interacting therewith, and FIGS. 5A through 5D illustrate examples of a user interface (UI) of an external device according to an embodiment. FIG. 6 is a block diagram illustrating a configuration of the fragrance discharge device of FIG. 1 and another external device interacting therewith.

Hereinafter, an interaction between the fragrance discharge device 10 and each of the external devices 20 and 22 is further described.

Referring to FIG. 4, the external device 20 according to an embodiment may be a mobile device such as a smartphone of a user. The external device 20 may include a UI 270 configured to receive user information, a sensor portion 260 configured to recognize environmental information around the external device 20 or the user information, a communicator 250 configured to communicate with the communicator 150 of the fragrance discharge device 10, a controller 210 configured to control the UI 270 and the communicator 250, and a memory 220 configured to store data.

The sensor portion 260 is configured to detect body information of the user or the environmental information around the external device 20 and may include at least one of a location sensor, an odor sensor, a heartrate sensor, a proximity sensor, an illuminance sensor, a temperature sensor, a sound sensor, an IR sensor, and an image sensor. Thus, the fragrance discharge device 10 may use environmental information around a space in which the fragrance discharge device 10 is present, which is measured through the sensor portion 260 of the external device 20, for example, information on an installation location, weather, illuminance, temperature, and an odor, in addition to information measured at the sensor portion 160 of the fragrance discharge device 10. Also, the fragrance discharge device 10 may use physical condition information, such as a heartrate of the user measured through the sensor portion 260, and information on psychology and emotions of the user, which is verified based on the measured physical condition information. The fragrance discharge device 10 may be controlled based on user profile information stored in the memory 220 of the external device 20. For example, the external device 20 may store, in the memory 220 as a database, a preference of the user on a specific fragrance material, an olfactory sensitivity, and body information such as a biorhythm, and may and may generate user profile information by combining the information. Also, the generated user profile information may be stored in a cloud server in addition to the memory 220 of the external device 20. Thus, other user devices communicably connectable to the fragrance discharge device 10 may use the fragrance discharge device 10 based on the user profile information.

The external device 20 may store, in the memory 220, context information including at least one of user information of the user using the fragrance discharge device 10 and environmental information around the fragrance discharge device 10 detected through the sensor portion 260, and, if necessary, may transmit the context information to the fragrance discharge device 10 through the communicator 250. The controller 110 of the fragrance discharge device 10 may control the discharge portion 140 based on the context information received from the external device 20 through the communicator 150.

The controller 210 is provided in a body of the external device 20 and may include, for example, a microprocessor. The controller 210 may control the sensor portion 260, the UI 270, and the communicator 250 of the external device 20.

If a remaining amount of the fragrance material stored in the storage 130 of the fragrance discharge device 10 is insufficient, the fragrance discharge device 10 may transmit an alert notification indicating the insufficiency of the remaining amount through the communicator 150 and the external device 20 may receive the alert notification through the communicator 250. The received alert notification may be displayed through the UI 270 of the external device 20. Accordingly, the user may opportunely become aware of fragrance material replacement information of the fragrance discharge device 10. Also, the user may control the fragrance discharge device 10 at a remote location through the external device 20. For example, when the user is to move from an outside to a room in which the fragrance discharge device 10 is installed, the user may control the fragrance discharge device 10 to discharge a desired fragrance material in advance through the UI 270 of the external device 20, such that the room may have a fresh atmosphere.

Although the present embodiment is described using the smartphone of the user as an example of the external device 20, it is provided as an example only. The external device 20 may be a multimedia player, for example, another mobile device such as a tablet PC, an MP3 player, a personal digital assistant (PDA), a virtual reality (VR) providing device such as a head mounted device (HMD), a smart band, and smart glasses. In this case, the fragrance discharge device 10 may control discharging of a fragrance material based on information on content being played on the multimedia player, for example, a scene of movie, lyrics, rhythm, and mood of music.

The UI 270 may be a touch display of the external device 20 in response to an operation of an application executed on the external device 20. The UI 270 may provide an alert notification to the user if a remaining amount of a fragrance material of the fragrance discharge device 10 is insufficient, and the user may perform an input through the UI 270 so that the fragrance discharge device 10 may operate in a predetermined mode. For example, the user may select a desired fragrance material through the UI 270. Also, the UI 270 may display information on a fragrance material and a type of a mode currently in operation, and a scheduled replacement period. If the fragrance discharge device 10 malfunctions, the UI 270 may display corresponding error information.

FIGS. 5A through 5D illustrate examples of a UI of an external device according to an embodiment.

Referring to FIG. 5A, the UI 270 of the external device 20 capable of interacting with a fragrance discharge device according to an embodiment is illustrated. The external device 20 refers to a smartphone of a user and includes all of the components of the external device 20 of FIG. 4. The UI 270 refers to an interface for an application executed on the smartphone of the user and the user may control the fragrance discharge device 10 using the UI 270. The UI 270 may include a fragrance material cartridge indicator 272 configured to mount to the fragrance discharge device 10, a remaining amount indicator 273 configured to indicate a remaining amount of a fragrance material, and a control setter 274 configured to set a control environment for discharging each fragrance material. In addition, although not illustrated, the UI 270 may provide an interface capable of displaying activity and biometric information of the user, emotion information, and the like. Through the UI 270, the user may select a setting for a single fragrance material, may select a setting for a blending method thereof, or may select a setting of the fragrance discharge device 10 based on a physical condition or an emotional state of the user according to a daily schedule of the user from a main menu 271.

Referring to FIG. 5B, the user may effectively select a desired blending ratio 276 through a mixing ratio selection window 275 on which blending ratios 276 for a plurality of fragrance materials are visually displayed. Areas representing two or more fragrance materials may be visually distinguishably displayed on the mixing ratio selecting window 275. A color in which colors representing the respective fragrance materials are mixed based on a ratio of each corresponding fragrance material may be displayed in a neighboring area between different fragrance materials. A ratio of each fragrance material may be calculated in response to the user clicking on a color representing the desired blending ratio 276. The calculated ratio of each fragrance material may be transmitted to the fragrance discharge device 10. The fragrance discharge device 10 may generate a fragrance desired by the user by selecting a suitable fragrance material based on the received ratio of each fragrance material and by controlling a discharge ratio thereof.

Referring to FIG. 5C, the user may select blending ratios for a plurality of fragrance materials through another interface screen. For example, based on a partition line 277c, a surface area of an upper area 277a may represent a relative amount of a first fragrance material and a surface area of a lower area 277b may represent a relative amount of a second fragrance material. The user may adjust a blending ratio of a desired fragrance material by clicking on and vertically moving the partition line 277c. Also, the user may select a fragrance material to be blended by clicking on a fragrance material selection button 278.

Referring to FIG. 5D, information on a physical condition or an emotional state of the user according to a daily schedule may be displayed on a smartphone display 279 of the user. Information on the physical condition or the emotional state of the user measured using various types of sensors mounted to the smartphone of the user. That is, information on an amount of time used to take a rest during the day and a state of the rest, an amount of time used to concentrate on a work during the day and strength of work, and an amount of time used to have a sleep during the day and a state 1 of the sleep may be stored and displayed. The user may set an operating time of the fragrance discharge device 10 based on the above information and may set a discharge point in time, a discharge pattern, and a blending type of a fragrance material such that the fragrance discharge device 10 may provide an olfactory environment suitable for the user.

Referring to FIG. 6, the external device 22 according to an embodiment refers to a device capable of discharging a fragrance material and may be a product functionally similar to the fragrance discharge device 10 of FIG. 1. The external device 22 is configured to effectively discharge a fragrance material within a predetermined space in conjunction with the fragrance discharge device 10.

In detail, the external device 22 may include a storage 230 configured to store a fragrance material, a body to which one or more storages 230 are mounted, a discharge portion 240 provided to the body and configured to discharge, to an outside, the fragrance material supplied from the storage 230, the controller 210 configured to control the discharge portion 240 to discharge, to the outside, the fragrance material stored in the storage 230, the UI 270 configured to receive user information, the sensor portion 260 configured to recognize environmental information around the external device 22 or the user information, and the communicator 250 configured to communicate with the communicator 150 of the fragrance discharge device 10. A primary function and role of the external device 22 are substantially identical to those of the fragrance discharge device 10. Therefore, further description related to the external device 22 is omitted herein and an interaction between the external device 22 and the fragrance discharge device 10 is further described.

The fragrance discharge device 10 and the external device 22 capable of interacting with the fragrance discharge device 10 and discharging a fragrance material may be provided within a predetermined space in which the user is present. In this case, the fragrance discharge device 10 and the external device 22 may interact with each other based on information on at least one of a physical location within the space, a relative distance from the user, and body information of the user. For example, when the user is relatively close to the fragrance discharge device 10 and relatively away from the external device 22, the fragrance discharge device 10 and the external device 22 may verify distances from the fragrance discharge device 10 and the external device 22 to the user through the sensor portions 160 and 260, respectively. Only the fragrance discharge device 10 relatively close to the user may discharge a specific fragrance material. Also, when it is verified that the user desires to discharge a large amount of fragrance material based on psychological information of the user or a selection of the user, all of the fragrance discharge device 10 and the external device 22 may operate to discharge the fragrance material. When two or more users are present in the same space, each user may control a relatively close device between the fragrance discharge device 10 and the external device 22 to discharge a desired fragrance material. Each of the fragrance discharge device 10 and the external device 22 may verify a user relatively close thereto through the corresponding sensor portion 160 or 260 and may discharge a fragrance material most suitable for the corresponding user.

In addition, although the fragrance discharge device 10 and the external device 22 are not present in the same space, the fragrance discharge device 10 and the external device 22 may be disposed in mutually neighboring spaces based on a movement line of the user, or may be physically separate from each other and disposed in a temporally continuous space. In this case, the fragrance discharge device 10 and the external device 22 may exchange mutual current states such that continuous emission may be provided to the user although the user moves.

Control Method of Fragrance Discharge Device

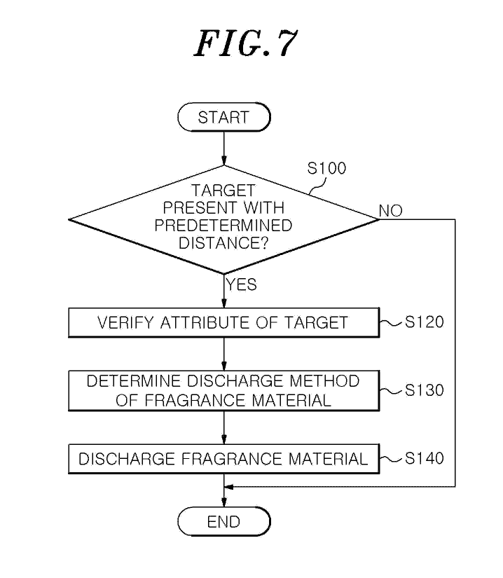

FIG. 7 is a flowchart illustrating a control method of a fragrance discharge device according to an embodiment.

Referring to FIGS. 1 and 7, in operation S100, the fragrance discharge device 10 verifies whether a target is present within a predetermined distance from the fragrance discharge device 10 through the sensor portion 160. Here, the sensor portion 160 may include at least one of a proximity sensor, an IR sensor, an odor sensor, and an image sensor. In operation S120, the controller 110 performs image processing based on information sensed through the sensor portion 160 and verifies an attribute of the target. In this case, the attribute of the target may information on at least one of a type of the target, a color of the target, a material of the target, a movement of the target, and an odor of the target. In operation S130, the controller 110 determines a discharge method of the fragrance material based on the verified attribute of the target. In operation S140, the controller 110 controls the discharge portion 140 to discharge the fragrance material based on the determined discharge method. Here, the discharge method of the fragrance material may determine at least one of a type of the fragrance material, an amount of the fragrance material, whether to discharge the fragrance material, a discharge direction, a discharge pattern, and a discharge location of the fragrance material relative to the target. For example, when a face of the user within a predetermined distance from the fragrance discharge device 10 is recognized as a location of the target through the sensor portion 160, the discharge direction may be changed such that the fragrance material may not be directly discharged toward the face of the user. Also, a type and intensity of the fragrance material may be changed depending on whether the target is a person or an animal. Also, when the target is the person, the fragrance material may be discharged by verifying a material of the target, for example, a material (leather, cloth) of clothes worn by the person or by adjusting a type, intensity, and/or a perfuming time of the fragrance material based on a size of the target. Also, the type of the fragrance material to be discharged may be changed depending on whether the target enters or leaves the space in which the fragrance discharge device 10 is present.

Referring to FIGS. 4 and 7, the fragrance discharge device 10 may verify whether the target is present within a predetermined distance from the fragrance discharge device 10 based on information received through the communicator 150 instead of using information sensed at the sensor portion 160. For example, near field communication may be connected between the fragrance discharge device 10 and the external device 20 of the user. Accordingly, when the user enters the space in which the fragrance discharge device 10 is present, the fragrance discharge device 10 may connect to the external device 20 of the user and may verify that the user has entered the space. In this case, the fragrance discharge device 10 may receive a profile of the user stored in the external device 20 of the user and may verify the attribute of the target based on a preferred fragrance material of the user or body information of the body S120 and may determine the discharge method of the fragrance material accordingly in operation S130. For example, if the user profile includes health anomaly information on an olfactory organ (nasal cavity, oral cavity) of the user, the fragrance discharge device 10 may select a fragrance material that does not irritate the olfactory organ of the user based on the information in operation S130 and may discharge the fragrance material in a direction that does not irritate the olfactory organ of the user in operation S140.

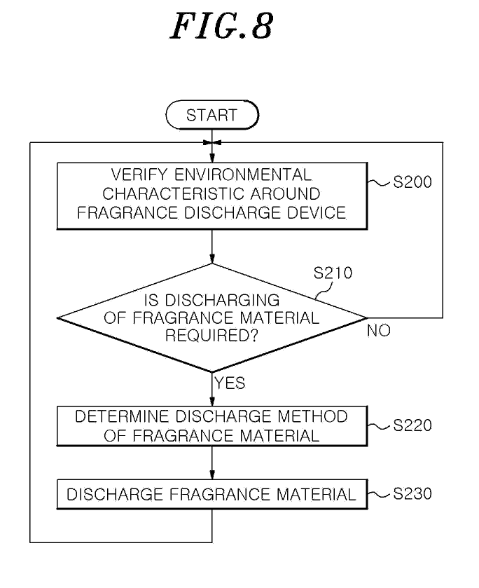

FIG. 8 is a flowchart illustrating a control method of a fragrance discharge device according to another embodiment.

Referring to FIGS. 1, 4, and 8, in operation S200, the fragrance discharge device 10 verifies environmental characteristics around the fragrance discharge device 10. In operation S210, the fragrance discharge device 10 determines whether discharging of the fragrance material is required based on the verified environmental characteristics. Here, the environmental characteristics around the fragrance discharge device 10 may include environmental characteristic information sensed at the sensor portion 160 or the sensor portion 260 of the external device 20 and environmental characteristic information received from the external device 20 of the user. The sensor portion 160 may include at least one of a proximity sensor, an IR sensor, an odor sensor, and an image sensor. The environmental characteristics may include at least one of spatial information including characteristics of the space in which the fragrance discharge device 10 is present, time information, and odor information. For example, when the user cooks in the space in which the fragrance discharge device 10 is present, the sensor portion 160 of the fragrance discharge device 10 may determine whether cooking is ongoing using the odor sensor. In this case, an appropriate fragrance material may be discharged to mask a smell of cooking in the corresponding space. When the fragrance discharge device 10 is disposed in a toilet, the fragrance discharge device 10 may detect an unpleasant smell by use of the toilet and may discharge a pleasant fragrance material such as a smell of coffee. When the user is a female and determined to be physiological, the external device 20 of the user may operate the fragrance discharge device 10 to block an unpleasant odor coming from the user before the user enters the space in which the fragrance discharge device 10 is installed, based on physiological cycle information input in advance from the user. Also, the external device 20 of the user may operate in advance the fragrance discharge device 10 to block an unpleasant smell of food from clothes of the user before the user having had a meal at a restaurant enters the space in which the fragrance discharge device 10 is present. In this case, the sensor portion 260 of the external device 20 of the user may verify whether the user has used a restaurant using a location sensor, such as a global positioning system (GPS). In operation S220, the controller 110 determines the discharge method of the fragrance material based on the verified environmental characteristics. In operation S230, the controller 110 controls the discharge portion 140 to discharge the fragrance material based on the determined discharge method. Although discharging of the fragrance material is completed, the fragrance discharge device 10 continuously detects a change in the environmental characteristics in operation S200. For example, the fragrance discharge device 10 detects a change in characteristic information of the space or characteristic information of the user, such as if the user leaves or cooks within the space, or if the user takes a rest or sleep. In this case, the fragrance discharge device 10 may verify spatial characteristics and user characteristics using the sensor portion 160 of the fragrance discharge device and may also use user characteristics or spatial characteristics measured by the sensor portion 260 of the external device 20 of the user. Also, the fragrance discharge device 10 may use user related information stored by the external device 20, for example, schedule information, physical condition information measured by the sensor portion 260 of the external device 20, psychological information of the user verified based on the measured physical condition information. If a new environmental characteristic is detected in operation S200, the fragrance discharge device 10 determines whether there is a need to discharge the new fragrance material based on the new environmental characteristic in operation S210. The fragrance discharge device 10 determines a discharge method of the new fragrance material based on the new environmental characteristic in operation S220 and the controller 110 controls the discharge portion 140 to discharge the new fragrance material based on the determined discharge method in operation S230.

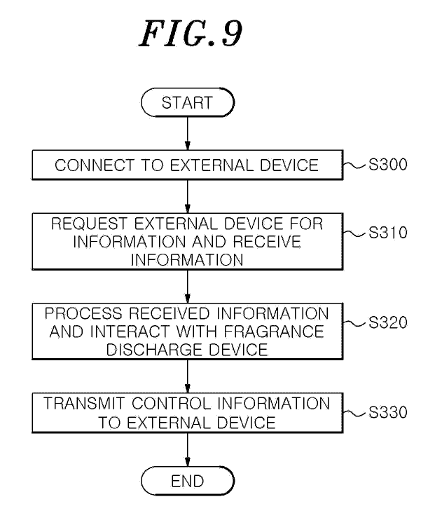

FIG. 9 is a flowchart illustrating a control method of a fragrance discharge device according to another embodiment. FIG. 9 is a flowchart illustrating an operation of actively controlling, by the fragrance discharge device 10, the external device 20 in conjunction with the external device 20.

Referring to FIGS. 1, 4, and 9, in operation S300, the fragrance discharge device 10 connects to the external device 20 communicably connectable to the fragrance discharge device 10. Here, the external device 20 refers to a device that may operate in conjunction with the fragrance discharge device 10 and may be one of, for example, a window inside a room, an air conditioner, an air cleaner, a drying machine, a light, a ventilator, a humidifier, and a vacuum cleaner. The fragrance discharge device 10 may connect to the external device 20 using IrDA, Bluetooth, ZigBee, or WiFi. Once the connection is completed, the fragrance discharge device 10 requests the external device 20 for external device related information and receives the same in operation S310. In this case, the external device 20 may be, for example, a window that may be opened and closed automatically. If the fragrance discharge device 10 is to ventilate the room by discharging a specific fragrance material in a space, the window may be closed in advance. When the fragrance discharge device 10 attempts a communication connection with an actuator of the window and connects thereto, a current state (open/closed) of the window may be received and the fragrance discharge device 10 may be activated based on the received state information in operation S320. That is, when the window is open, the fragrance discharge device 10 may transmit control information for closing the window to the external device 20 in operation S330 and may transmit control information for opening the window to ventilate the room to the external device 20 before discharging the fragrance material in operation S330.

As another example, the external device 20 may be a light. When the user is to have a sleep in a room, the fragrance discharge device 10 may discharge a fragrance material capable of inducing the user to have a good sleep. In this case, the fragrance discharge device 10 may attempt a communication connection with the light and may receive current state information of the light so that the user may have a good sleep. Based on the received state information of the light, the fragrance discharge device 10 may adjust a brightness of the light to be dark or may turn OFF the light the light is bright, and, conversely, may turn ON the light again after a preset period of time is elapsed. An action of the user to have a sleep may be verified from another device, such as a smartphone of the user interacting with the fragrance discharge device 10. For example, the smartphone of the user capable of detecting a biorhythm of the user may detect the biorhythm, may verify the action of the user to have a sleep, and may transmit the corresponding information to the fragrance discharge device 10. In this case, the fragrance discharge device 10 may discharge a fragrance material suitable for the user to have a sleep based on the received information and, at the same time, may change an illuminance of the wirelessly connected light inside the room to be dark.

As another example, when an amount of a fragrance material stored in the storage 130 of the fragrance discharge device 10 is insufficient, the fragrance discharge device 10 may operate in conjunction with conditioning equipment, such as an air conditioner or a humidifier, to provide a further effect olfactory environment in a space in which the user is present. That is, the fragrance discharge device 10 attempts a communication connection with an air conditioner or a humidifier that is the external device 20 in operation S300. Once the connection is completed, the fragrance discharge device 10 requests and receives air conditioner or humidifier related information in operation S310. The fragrance discharge device 10 may operate based on the received state information of the air conditioner or the humidifier in operation S320, and may control the air conditioner or the humidifier such that the fragrance material may be further effectively diffused in the space in operation S330.

As another example, the external device 20 may be a multimedia player such as a television (TV), an audio system, and a headset. The fragrance discharge device 10 may play the multimedia player to provide a further effective olfactory environment while discharging a fragrance material.

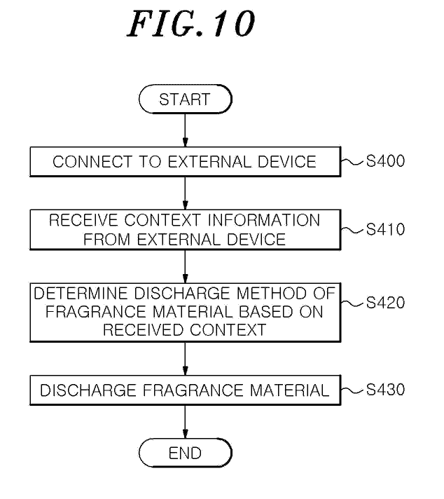

FIG. 10 is a flowchart illustrating a control method of a fragrance discharge device according to another embodiment. FIG. 10 is a flowchart illustrating an operation of performing, by the fragrance discharge device 10, discharging of a fragrance material based on context information received from the external device 20 in conjunction with the external device 20.

Referring to FIGS. 1, 4, and 10, in operation S400, the fragrance discharge device 10 connects to the external device 20 communicably connectable to the fragrance discharge device 10. Here, the external device 20 refers to a device that may operate in conjunction with the fragrance discharge device 10 and may be one of, for example, a window inside a room, an air conditioner, an air cleaner, a drying machine, a light, a ventilator, a humidifier, a vacuum machine, and a mobile device such as a smartphone of the user. Hereinafter, a description is made based on an example in which the external device 20 is the smartphone of the user. In operation S400, the fragrance discharge device 10 communicably connects to the smartphone of the user. The fragrance discharge device 10 may connect to the smartphone using IrDA, Bluetooth, ZigBee, or WiFi. Once the connection is completed, the fragrance discharge device 10 requests and receives external device related information in operation S410. In this case, the fragrance discharge device 10 may attempt the connection and, also, the smartphone may attempt to connect to the fragrance discharge device 10. In operation S410, the fragrance discharge device 10 may receive context information from the smartphone. In this case, the context information may include information associated with a psychological state and a body of the user. For example, when the user is detected to lie in bed during a preset period of time, the fragrance discharge device 10 may determine an appropriate fragrance material based on state (psychological state and physical strength) information of the user in operation S420 and the controller 110 may control the discharge portion 140 to discharge the fragrance material in operation S430. As another example, the external device 20 may be a light. If the light 20 changes into soft glow in a space in which the user is present, the fragrance discharge device 10 may determine and discharge a suitable fragrance material so that an atmosphere suitable for the light glow may be achieved. In addition, the external device 20 may be another device associated with the atmosphere in the space in which the user is present, for example, one of a curtain, an audio, a television, an air conditioner, a humidifier, and an air cleaner.

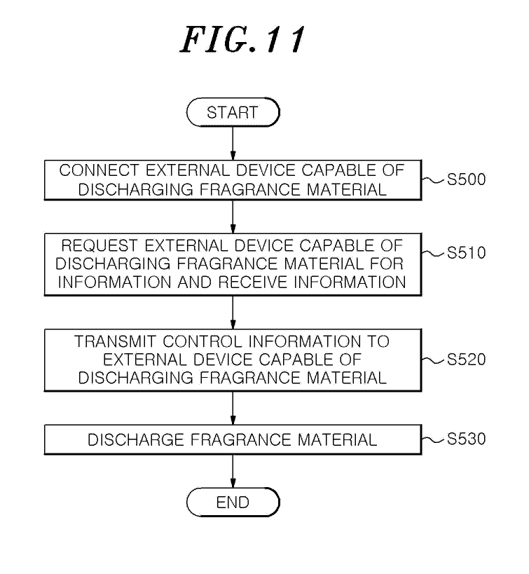

FIG. 11 is a flowchart illustrating a control method of a fragrance discharge device according to another embodiment. FIG. 11 is a flowchart illustrating an operation of performing, by the fragrance discharge device 10, discharging of a fragrance material in conjunction with the external device 22 capable of discharging the fragrance material.

Referring to FIGS. 1, 6, and 11, in operation S500, the fragrance discharge device 10 connects to the external device 22 communicably connectable and capable of discharging a fragrance material. The external device 22 refers to a device that may discharge the fragrance material and may be a product functionally similar to the fragrance discharge device 10. The fragrance discharge device 10 may connect to the external device 22 using IrDA, Bluetooth, ZigBee, or WiFi. Once the connection is completed, the fragrance discharge device 10 requests the external device 22 for external device related information and receives the same in operation S510. In this case, information associated with the external device 22 may include at least one of a location of the external device 22, a type and an amount of the fragrance material stored in the storage 230 of the external device 22, and user information and spatial information sensed at the sensor portion 260 of the external device 22. The fragrance discharge device 10 may determine a fragrance material discharge method within the space based on at least one of the received information associated with the external device 22, user information and spatial information sensed at the sensor portion 160, and type and amount information of the fragrance material stored in the storage 130 and may transmit control information associated with fragrance material discharging of the external device 22 in operation S520, and may control the discharge portion 140 of the fragrance discharge device 10 to discharge the fragrance material in operation S530. The external device 22 controls the discharge portion 240 to discharge the fragrance material based on the control information received from the fragrance discharge device 10.

### Example of Using Fragrance Discharge Device

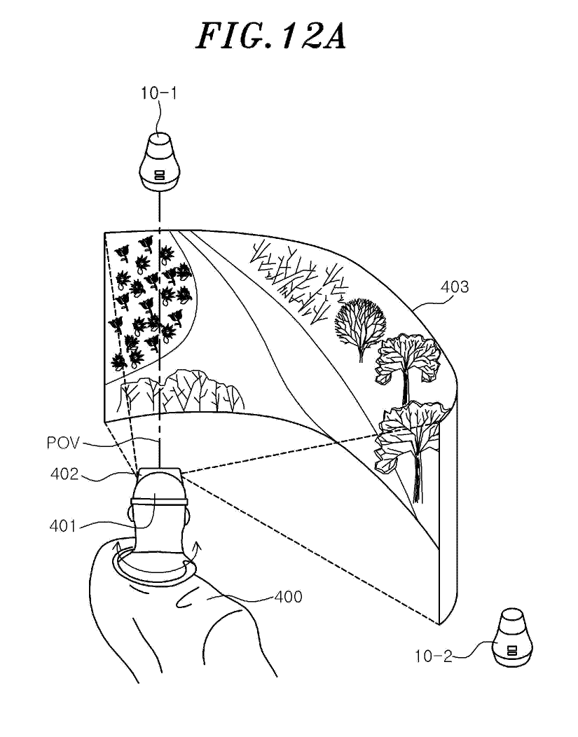

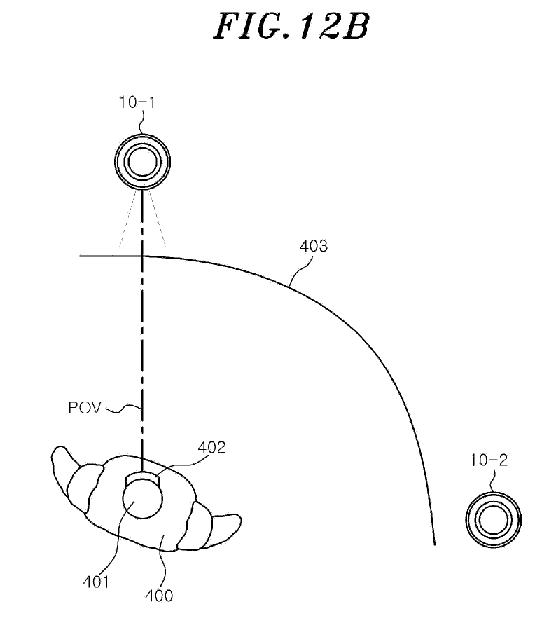

FIGS. 12A and 12B illustrate examples in which a fragrance discharge device interacts with a multimedia player according to an embodiment.

Referring to FIGS. 12A and 12B, a user 400 is wearing a head mounted device (HMD) 402 around a head 401. Fragrance discharge devices 10-1 and 10-2 are disposed on the left and the right of the user 400, respectively. Each of the fragrance discharge devices 10-1 and 10-2 includes a configuration of the fragrance discharge device 10 of FIG. 4. Each of the fragrance discharge devices 10-1 and 10-2 may communicably connect to the HMD 402. The HMD 402 includes a configuration of the external device 20 of FIG. 4. The user 400 may experience virtual reality through the HMD 402. A screen 403 refers to a virtual reality image provided to the user 400 through the HMD 402 worn by the user 400 and tracks a point of view (POV) of the user 400 and displays a screen corresponding to the POV in response to rotating the head 401 of the user 400. Referring to FIG. 12A, if the user 400 turns the head 401 to the left, the POV of the user 400 faces a flowerbed displayed on the left of the screen 403. In this case, the fragrance discharge device 10-1 close to the POV of the user 400 may operate by interacting with the HMD 402. The HMD 402 may analyze an image of the flowerbed currently present on the POV of the user 400 and may transmit corresponding information to the fragrance discharge device 10-1. The fragrance discharge device 10-1 may discharge a suitable fragrance material, for example, a rose fragrance, toward the user 400 based on image information received from the HMD 402. That is, the fragrance discharge device 10-1 may determine olfactory environmental information associated with an image corresponding to virtual location information on a virtual reality space on which the POV of the user 400 is present, which varies in response to a movement of the head 401 of the user 400, and may discharge the suitable fragrance material. In this case, the fragrance discharge device 10-1 may analyze information of the user 400 sensed at the sensor portion 160 of the fragrance discharge device 10-1, may verify a location of an olfactory organ (lips, nose) of the user 400, and may dispense the fragrance material toward the verified olfactory organ at appropriate intensity. In the meantime, if the POV of the user 400 directs the right, that is, a tree on the screen 403, the fragrance discharge device 10-2 closest to the POV of the user 400 may operate by interacting with the HMD 402. The fragrance discharge device 10-2 may discharge a suitable fragrance material, for example, a fragrance material emitting a forest fragrance, toward the user 400 based on image information received from the HMD 402. Also, the fragrance discharge devices 10-1 and 10-2 may simultaneously discharge suitable fragrance materials based on the overall atmosphere of an image displayed on the HMD 402, regardless of the POV of the user 400. When the user 400 is into a game situation without moving during a preset period of time or more in a game space using the HMD 402, it is possible to increase a degree of immersion of the user 400 into the game by discharging a fragrance material suitable for an image of a corresponding section displayed on the HMD 402. Also, a variety of sensors, for example, an electrocardiogram (ECG) sensor, an electroencephalogram (EEG) sensor, a photo-plethysmography (PPG) sensor, a blood oxygen saturation level (S_{P}O₂) sensor, a brainwave sensor, and an image sensor, may be mounted to the HMD 402. The HMD 402 may verify an emotion or a feeling of the user 400 using the sensor(s) mounted to the HMD 402 and may transmit corresponding information to the fragrance discharge devices 10-1 and 10-2 to discharge a fragrance material suitable for the current emotion or feeling of the user 400.

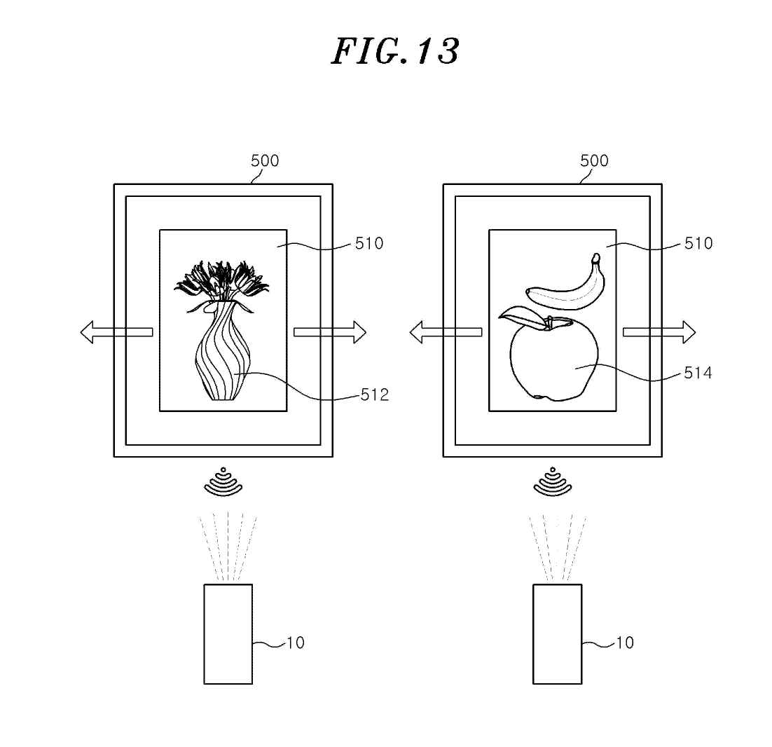

FIG. 13 illustrates an example in which a fragrance discharge device interacts with another multimedia player according to an embodiment.

Referring to FIG. 13, each of digital pictures 512 and 514 is displayed on an image frame 510 within a digital frame 500. At least one fragrance discharge device 10 may be disposed around the digital frame 500. The fragrance discharge device 10 may include a configuration of the fragrance discharge device 10 of FIG. 4 and the digital frame 500 may include a configuration of the external device 20 of FIG. 4. The fragrance discharge device 10 and the digital frame 500 may operate through a mutual interaction. That is, the digital frame 500 may transmit, to the fragrance discharge device 10, information associated with an image currently displayed on the digital frame 500. The fragrance discharge device 10 may select a suitable fragrance material based on the received image information and may discharge the selected fragrance material around the digital frame 500. If an image of the digital frame 500 changes from a flower of the digital picture 512 to a fruit of the digital picture 514, the fragrance discharge device 10 may discharge a fragrance material of a fruit fragrance while discharging a fragrance material of a flower fragrance in response to the change. Meantime, the fragrance discharge device 10 may receive information on a psychological state or a health state of a user in conjunction with a mobile device such as a smartphone of the user. The fragrance discharge device 10 may select a suitable fragrance material based on the received information on the psychological state or the health state and may discharge the selected fragrance material around the digital frame 500. At the same time, the fragrance discharge device 10 may transmit information on the psychological state or the health state of the user to the digital frame 500 so that a suitable image may be displayed on the image frame 510 of the digital frame 500. Herein, it is described as an example that the digital frame 500 and the fragrance discharge device 10 are provided as separate configurations. However, the fragrance discharge device 10 may be provided in the digital frame 500.

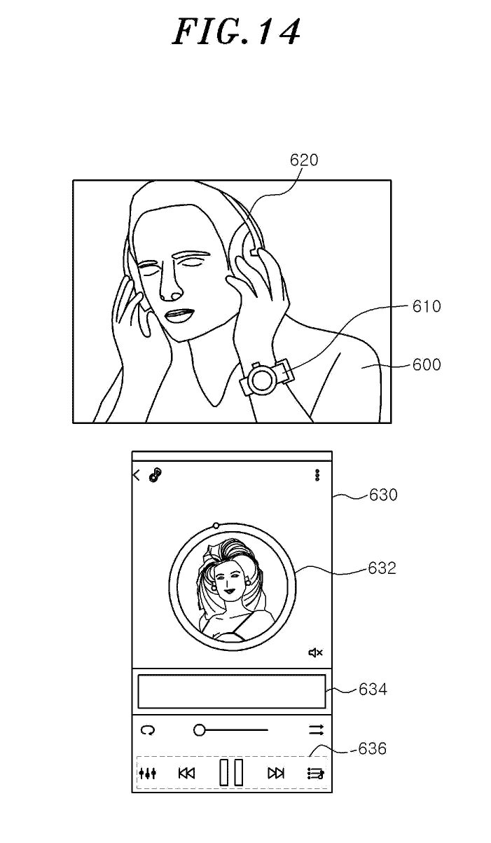

FIG. 14 illustrates an example in which a fragrance discharge device interacts with an external device according to an embodiment.

Referring to FIG. 14, a user 600 is wearing a headset 620 around a head and also wearing a smartwatch 610 around a wrist. A variety of sensors, for example, an ECG sensor, an EEG sensor, a PPG sensor, an S_{P}O₂ sensor, a brainwave sensor, and an image sensor may be mounted to the headset 620. Also, a variety of sensors capable of measuring ECG or a psychological state of the user 600 may be mounted to the smartwatch 610. The user 600 may listen to music playing on a multimedia player 630 of the user 600 through the headset 620. The multimedia player 630 may include jacket information 632 on a currently playing album, lyrics information 634 on lyrics of a currently playing song, and control information 636 used to select music to be played and control sound effect. Although not illustrated, a fragrance discharge device may be provided around the user 600. The fragrance discharge device may include a configuration of the fragrance discharge device 10 of FIG. 4 and the multimedia player 630 may include a configuration of the external device 20 of FIG. 4. To enhance a degree of immersion on music of the user 600 listening to the music playing on the multimedia player 630 through the headset 620, the multimedia player 630 may transmit information associated with the playing music, for example, the jacket information 632, a music bit, a music style, and the lyrics information 634 to the fragrance discharge device 10. The fragrance discharge device 10 may select and discharge a fragrance material capable of enhancing the degree of immersion of the user 600 based on the received music related information. Physical condition information of the user 600 may be measured through the variety of sensors mounted to the headset 620 and the variety of sensors mounted to the smartwatch 610, and information associated with a psychological state and a health state of the user 600 may be verified based on the measured physical condition information. Additionally, information associated with the psychological state and the health state of the user 600 may be verified based on social media activity information (Facebook, Twitter) and schedule information of the user 600. The headset 620 or the smartwatch 610 may transmit, to the fragrance discharge device 10, psychological state information that is verified based on the detected heath state of the user 600 or specified physical condition information of the user 600. The fragrance discharge device 10 may discharge a suitable fragrance material based on the received user physical condition information and psychological state information. If a plurality of users 600 is listening to the same music within the same space, the fragrance discharge device 10 may select and discharge a suitable fragrance material based on average heath state or psychological state information of the plurality of users 600 present within the space. If music playing on the multimedia player 630 changes, the fragrance discharge device 10 may appropriately change and discharge a type, a discharge direction, intensity, etc., of a fragrance material to be discharged. Also, the multimedia player 630 may measure physical condition information of the user 600 with respect to the fragrance material that is discharged from the fragrance discharge device 10 using the sensors mounted to the headset 620 or the sensors mounted to the smartwatch 610, and may receive psychological state information of the user 600 that is verified based on the measured physical condition information of the user and may store the same as olfactory environmental information of the user 600. Accordingly, if the same user 600 subsequently listens to the same music again through the multimedia player 630, the fragrance discharge device 10 may discharge the previous fragrance material based on the stored olfactory environmental information of the user 600 and may provide the user 600 with the degree of immersion the user 600 has previously perceived. Although it is described herein as an example that the multimedia player 630 is a music player, the same effect may be achieved when the multimedia player 630 is a smartphone of the user 600, a TV, a PDA, a laptop computer, a PC, or a digital camera. For example, the user 600 may wear the smartwatch 610 capable of measuring physical condition information of the user 600 and verifying an emotion or a feeling of the user based on the measured physical condition information and, in this state, may capture an image of an object using a digital camera capable of interacting with the smartwatch 610. In this case, the smartwatch 610 may verify and store a health state and a psychological state of the user at a capturing point in time or an atmosphere around a place where capturing is performed. Subsequently, when the user 600 views the corresponding image using the multimedia player 630, the fragrance discharge device 10 interacting with the multimedia player 630 may determine and discharge a fragrance material based on the stored health state or psychological state of the user 600 or atmosphere information around the space, so that the user 600 may be immersed into the atmosphere at the time of capturing.

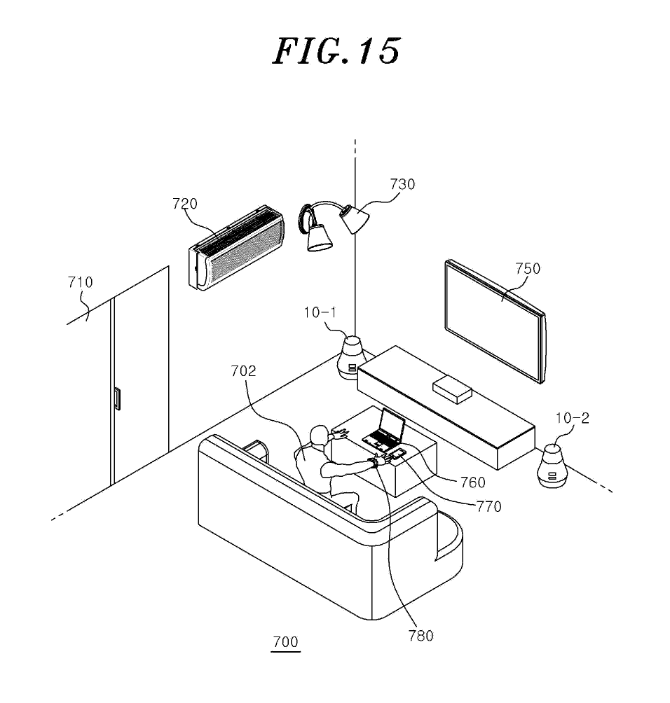

FIG. 15 illustrates an example in which a fragrance discharge device interacts with various external devices according to an embodiment.

Referring to FIG. 15, a plurality of fragrance discharge devices 10-1 and 10-2 and various external devices 710, 720, 730, 750, 760, 770, and 780 are present within a space 700 in which a user 702 is present. The fragrance discharge device 10-1 may include a configuration of the fragrance discharge device 10 of FIG. 4, and the fragrance discharge device 10-2 may include a configuration of the fragrance discharge device 22 of FIG. 6. Also, each of the external devices 710, 720, 730, 750, 760, 770, and 780 may include a configuration of the external device 20 of FIG. 4. The fragrance discharge devices 10-1 and 10-2 and the external devices 710, 720, 730, 750, 760, 770, and 780 may connect to each other to be directly communicable using WiFi, ZigBee, IrDA, Bluetooth communication, and the like. For example, the fragrance discharge device 10-1 may function as a master to control the fragrance discharge device 10-2 and the external devices 710, 720, 730, 750, 760, 770, and 780. Also, the fragrance discharge devices 10-1 and 10-2 and the external devices 710, 720, 730, 750, 760, 770, and 780 may be mutually communicably connected over an Internet network using an access point (not shown). The fragrance discharge device 10-1 may control discharging of a fragrance material within the space 700 based on characteristic information of the space 700 sensed using the fragrance discharge device 10-2 and the various external devices 710, 720, 730, 750, 760, 770, and 780 and characteristic information of the user 702. For example, the fragrance discharge device 10-1 may detect an open/closed state of the window 710 and may control opening/closing of the window 710, and may control diffusion of a fragrance material n conjunction with the air conditioner 720. Also, the fragrance discharge device 10-1 may verify the atmosphere of the space 700 based on a lighting state of the light 730 and may discharge a suitable fragrance material based on the verified atmosphere of the space 700 in conjunction with the fragrance discharge device 10-2. Each of the fragrance discharge devices 10-1 and 10-2 may discharge a suitable fragrance material at suitable intensity based on a relatively location of the user 702 within the space 700. The fragrance discharge devices 10-1 and 10-2 may receive multimedia content information playing on the television 750, the laptop PC 760, and the smartphone 770, and may receive physical condition information of the user 702 measured through various types of biometric sensors mounted to the smartwatch 780 worn by the user 702 and psychological state information that is verified based on the measured physical condition information. The fragrance discharge devices 10-1 and 10-2 may discharge a fragrance material based on the received information so that the user 702 may be further immersed into multimedia contents.

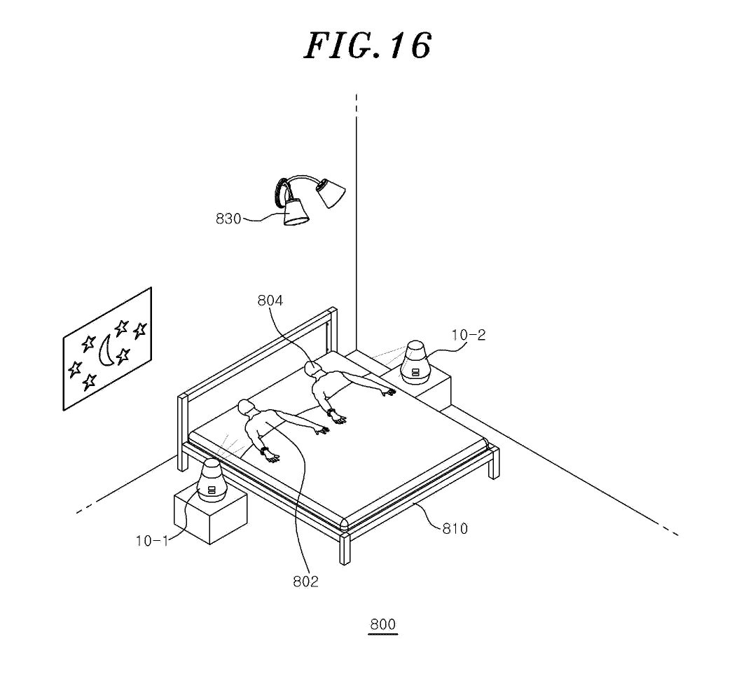

FIG. 16 illustrates another example in which a fragrance discharge device interacts with various external devices according to an embodiment.

Referring to FIG. 16, a plurality of users 802, 804, for example, a first user 802 and a second user 804, lie in bed 810 within a space 800. Here, it is a nighttime and the users 802 and 804 are to have a sleep by adjusting a light 830 to be dark. The fragrance discharge device 10-1 and the fragrance discharge device 10-2 are disposed at both sides of the bed 810, respectively. Each of the fragrance discharge devices 10-1 and 10-2 may include a configuration of the fragrance discharge device 10 of FIG. 1. Each of the fragrance discharge devices 10-1 and 10-2 may detect a location and a state of the corresponding user 802, 804 closest thereto through an IR sensor or an image sensor of the sensor portion 160 thereof. For example, when the users 802 and 804 are lying in bed 810 during a preset period of time or more, the fragrance discharge devices 10-1 and 10-2 may verify whether the corresponding users 802 and 804 are to take a sleep and may discharge a fragrance material suitable for sleep, respectively. In this case, each of the fragrance discharge devices 10-1 and 10-2 may discharge a fragrance material desired by each corresponding user 802 or 804 while sleeping based on a pre-stored user profile of each corresponding user 802 or 804. Also, the fragrance discharge device 10-1 may adjust a discharge direction and intensity of a fragrance material so that the fragrance material may be discharged only toward the user 802 relatively close to the fragrance discharge device 10-1 in order to transfer the fragrance material only the user 802 and to minimize transferring of the fragrance material to the user 804. Likewise, the fragrance discharge device 10-2 may adjust a discharge direction and intensity of a fragrance material so that the fragrance material may be discharged only toward the user 804 relatively close to the fragrance discharge device 10-2 in order to transfer the fragrance material only to the user 804 and to minimize transferring of the fragrance material to the user 802. Also, each of the fragrance discharge devices 10-1 and 10-2 may control discharging of the fragrance material using state information of the light 830 in addition to information acquired using the sensor portions 160 and 260. For example, if the users 802 and 804 lie in bed 810 and the light 830 is turned ON to be bright, the fragrance discharge devices 10-1 and 10-2 may verify that the users 802 and 804 do not desire to take a sleep and may discharge different types of fragrance materials. Also, if the user 802 is sleeping and the user 804 is to wake up for reading, the fragrance discharge device 10-1 may discharge a fragrance material suitable for sleep and the fragrance discharge device 10-2 may discharge a fragrance material suitable for immersion into reading.

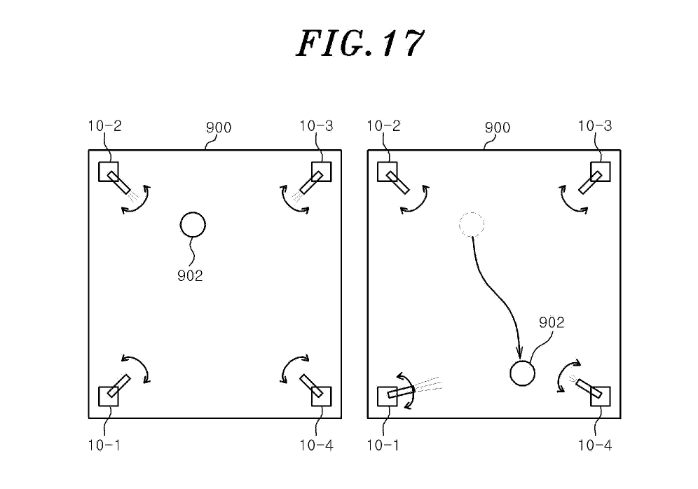

FIG. 17 illustrates an example in which a plurality of fragrance discharge devices operates through mutual interaction according to an embodiment.

Referring to FIG. 17, four fragrance discharge devices 10-1, 10-2, 10-3, and 10-4 are present at corners within a space 900, respectively. The four fragrance discharge devices 10-1, 10-2, 10-3, and 10-4 may communicably connect to each other. Also, each of the four fragrance discharge devices 10-1, 10-2, 10-3, and 10-4 may adjust a direction of a nozzle in which a fragrance material is discharged in any direction. Each of the fragrance discharge devices 10-1, 10-2, 10-3, and 10-4 may include a configuration of the fragrance discharge device 10 of FIG. 1. Accordingly, each of the fragrance discharge devices 10-1, 10-2, 10-3, and 10-4 may verify environmental information around the space 900, such as a size and a shape of the space 900, and user information, such as a relative location of a target, that is, a user 902 and clothes and a size of the user 902 within the space 900 using the sensor portion 160, for example, an IR sensor, a laser sensor, an odor sensor, an image sensor, an ultrasonic sensor, a Bluetooth Low Energy (BLUE) sensor, and the like. Each of the fragrance discharge devices 10-1, 10-2, 10-3, and 10-4 may transmit the verified information to another fragrance discharge device and the other fragrance discharge device may control discharging of a fragrance material based on the received information. Referring to a figure on the left of FIG. 17, the user 902 is relatively close to the fragrance discharge device 10-2 and the fragrance discharge device 10-3 and accordingly, each of the fragrance discharge devices 10-2 and 10-3 discharges a suitable fragrance material toward the user 902. Here, the fragrance discharge devices 10-1 and 10-4 relatively away from the user 902 may not discharge a fragrance material. Referring to a figure on the right of FIG. 17, if the user 902 moves within the space 900 and becomes relatively close to the fragrance discharge device 10-1 and the fragrance discharge device 10-4, each of the fragrance discharge devices 10-1 and 10-4 starts to discharge a suitable fragrance material toward the user 902. Here, each of the fragrance discharge devices 10-2 and 10-3 stops discharging of the fragrance material. As described above, the fragrance discharge devices 10-1, 10-2, 10-3, and 10-4 disposed at predetermined locations within the space 900 may interact with each other and may verify the environmental information around the space 900 and the user information and share the information. Accordingly, a further effective performing effect within the space 900 may be achieved.

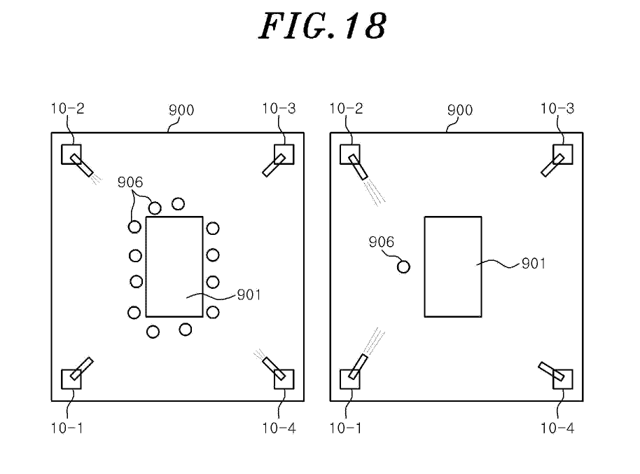

FIG. 18 illustrates another example in which a plurality of fragrance discharge devices operates through mutual interaction according to an embodiment.

Referring to FIG. 18, the four fragrance discharge devices 10-1, 10-2, 10-3, and 10-4 are present at corners within the space 900, respectively. The four fragrance discharge devices 10-1, 10-2, 10-3, and 10-4 may communicably connect to each other. Also, each of the four fragrance discharge devices 10-1, 10-2, 10-3, and 10-4 may adjust a direction of a nozzle in which a fragrance material is discharged in any direction. Each of the fragrance discharge devices 10-1, 10-2, 10-3, and 10-4 may include a configuration of the fragrance discharge device 10 of FIG. 1. Accordingly, each of the fragrance discharge devices 10-1, 10-2, 10-3, and 10-4 may verify environmental information around the space 900, such as a size and a shape of the space 900, and user information, such as a relative location of a target, that is, a user 906, clothes and a size of the user 906 within the space 900 using the sensor portion 160, for example, an IR sensor, a laser sensor, an odor sensor, an image sensor, an ultrasonic sensor, a BLE sensor, and the like. Referring to a figure on the left of FIG. 18, a table 901 is disposed at the center of the space 900 and a plurality of users 906 gathers around the table 901 and is having a conference. Each of the fragrance discharge devices 10-1, 10-2, 10-3, and 10-4 may verify that the users 906 are gathered within the space 900 through its sensor portion. Also, the fragrance discharge devices 10-1, 10-2, 10-3, and 10-4 may interact with smartphones of the respective corresponding users 906 and may receive user profiles thereof from the smartphones, respectively. Accordingly, each of the fragrance discharge devices 10-1, 10-2, 10-3, and 10-4 may receive data on fragrance materials preferred by the respective users 906 and may determine a most suitable fragrance material for the users 906. For example, the most suitable fragrance material may be determined based on an average of preferred fragrance materials of the users 906. The fragrance discharge devices 10-4 and 10-2 storing the corresponding fragrance material may discharge the corresponding fragrance material toward the users 906 based on the determined fragrance material. Here, if the corresponding fragrance material is stored in all of the fragrance discharge devices 10-1, 10-2, 10-3, and 10-4, only a fragrance discharge device closest to a center point of the gathered users 906 may operate. Also, intensity or cycle at which the fragrance material is discharged may be modified based on a number of the users 906. Also, a moisture ratio in the air within the space 900 may be measured through an air cleaner (not shown) provided in the space 900 and capable of interacting with the fragrance discharge device 10-1, 10-2, 10-3, and 10-4. If the moisture ratio in the air is less than or equal to a preset value, the fragrance discharge device 10-1, 10-2, 10-3, and 10-4 may select and discharge a fragrance material for increasing the moisture ratio. Referring to a figure on the right of FIG. 18, a large number of persons have left and only a single user 906 is present within the space 900. In this case, the fragrance discharge devices 10-1 and 10-2 relatively closest to the user 906 may discharge a fragrance material toward the user 906.

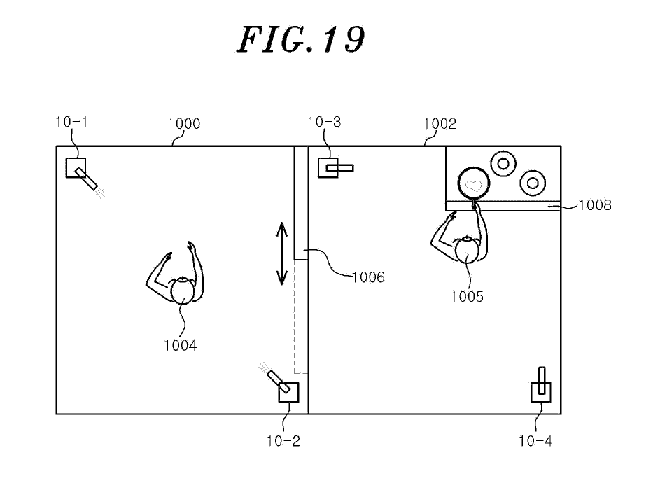

FIG. 19 illustrates another example in which a plurality of fragrance discharge devices operates through mutual interaction according to an embodiment.

Referring to FIG. 19, two spaces, for example, a first space 1000 and a second space 1002, are adjacent to each other and are separate by a door 1006. The two spaces 1000 and 1002 may communicate with each other or may be blocked through sliding of the door 1006. Herein, description is made based on an example in which the first space 1000 is a living room and the second space 1002 is a kitchen. Two fragrance discharge devices 10-1 and 10-2 are disposed at both corners to diagonally face each other within the first space 1000 and two fragrance discharge devices 10-3 and 10-4 are disposed at both corners to diagonally face each other within the second space 1002. The fragrance discharge devices may communicably connect to each other. Also, the fragrance discharge devices 10-1, 10-2, 10-3, and 10-4 may communicably connect to each other and may be automatically opened and closed by an actuator (not shown). Also, each of the four fragrance discharge devices may adjust a direction of a nozzle in which a fragrance material is discharged in any direction. Each of the fragrance discharge devices may include a configuration of the fragrance discharge device 10 of FIG. 1. Accordingly, each of the fragrance discharge devices may verify environmental information around the corresponding space 1000 or 1002, such as a size and a shape of the space 1000 or 1002, and user information, such as a relative location of a corresponding target, that is, a user 1004 or 1005, clothes and a size of the user 1004 or 1005 within the space 1000 or 1002 using its sensor portion 160, for example, an IR sensor, a laser sensor, an odor sensor, an image sensor, an ultrasonic sensor, a BLE sensor, and the like. The user 1004 within the first space 1000 is taking a rest and the user 1005 within the second space 1002 is cooking using a gas stove 1008. At an initial stage, the door 1006 is open to open the first space 1000 and the second space 1002. Once the user 1005 within the second space 1002 starts cooking, the fragrance discharge devices 10-3 and 10-4 disposed within the second space 1002 may verify that the cooking is ongoing within the second space 1002. In detail, each of the fragrance discharge devices 10-3 and 10-4 may verify that the cooking is ongoing within the second space 1002 using the odor sensor of its sensor portion 160. Alternatively, each of the fragrance discharge devices 10-3 and 10-4 may communicably connect to the gas stove 1008 and verify that the cooking is ongoing if the gas stove 1008 is ON during a preset period of time or more. If the user 1005 within the second space 1002 starts cooking, a smell of cooking may be spread into the first space 1000 and may cause the user 1004 taking a rest within the first space 1000 to feel unpleasant. To prevent this, when the fragrance discharge devices 10-3 and 10-4 detect that the cooking is ongoing within the second space 1002, the fragrance discharge devices 10-3 and 10-4 may transmit corresponding information to the fragrance discharge devices 10-1 and 10-2 within the first space 1000 and may operate the door 1006 to block the first space 1000 and the second space 1002 from each other. Each of the fragrance discharge devices 10-1 and 10-2 within the first space 1000 may discharge a fragrance material suitable for the user 1004 toward the first space 1000 so that the user 1004 may take a further comfortable rest. Also, when the cooking is completed within the second space 1002, each of the fragrance discharge devices 10-3 and 10-4 may discharge a suitable fragrance material toward the second space 1002 to remove a remaining smell of food and to refresh the second space 1002 to be atmospherically pleasant. Also, when the cooking is not ongoing within the second space 1002 and the door 1006 is opened such that the first space 1000 and the second space 1002 are opened to each other, the four fragrance discharge devices 10-1, 10-2, 10-3, and 10-4 may interact with each other and may discharge a suitable fragrance material over the entire first space 1000 and second space 1002.

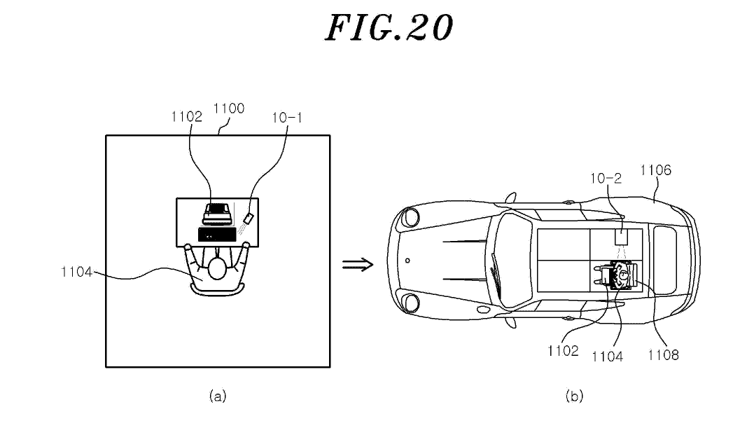

FIG. 20 illustrates another example in which a plurality of fragrance discharge devices operates through mutual interaction according to an embodiment. FIG. 20 illustrates an example of generating an olfactory environment similar to that of a previous space in another space when a user 1104 performs the same work in the other space.

Referring to FIG. 20, the user 1104 is carrying out a work using a laptop computer 1102 in an office 1100 of the user 1104. In this case, the fragrance discharge device 10-1 is disposed in the office 1100. The user 1104 may consecutively carry out the same work that has been carried out in the office 1100 while moving in a vehicle 1106. The fragrance discharge device 10-2 is disposed in the vehicle 1106. Each of the fragrance discharge devices 10-1 and 10-2 may include a configuration of the fragrance discharge device 10 of FIG. 1. Each of the fragrance discharge devices 10-1 and 10-2 may verify environmental information around the corresponding space 1100 or 1106, such as a size and a shape of the space, and user information, such as a relative location of a target, that is, the user 1104, clothes and a size of the user 1104 using its sensor portion 160, for example, an IR sensor, a laser sensor, an odor sensor, an image sensor, an ultrasonic sensor, a BLE sensor, and the like. The two fragrance discharge devices 10-1 and 10-2 may share mutual information through Internet connection and the like. In detail, the fragrance discharge devices 10-1 and 10-2 may store the verified user information in a cloud server through Internet connection. User information on the user 1104 verified by the fragrance discharge device 10-1 may be stored in the cloud server and when the fragrance discharge device 10-2 detects the user 1104, the fragrance discharge device 10-2 may download corresponding information from the cloud server and control discharging of a fragrance material based on the corresponding information. Also, each of the fragrance discharge devices 10-1 and 10-2 may communicably connect to the laptop computer 1102 of the user 1104 and may verify details of the work being carried out by the user 1104. The fragrance discharge device 10-1 may determine and discharge a suitable fragrance material to generate an optimal environment in which the user 1104 may concentrate on the work while the user 1104 is carrying out the work in the office 1100. Discharge information of the determined fragrance material may be transmitted to the cloud server. When the user 1104 moves to the vehicle 1106 and continues the work having been carried out in the office 1100, the fragrance discharge device 10-2 disposed in the vehicle 1106 may detect the user 1104 and verify the work being carried out through communication with the laptop computer 1102 of the user 1104. When the user 1104 is determined to continue the work having been carried out in the office 1100, the second fragrance discharge device 10-2 may connect to the cloud server, may download previous fragrance material discharge information, and may discharge the corresponding fragrance material. Accordingly, although a work space is changed, a degree of concentration of the user 1104 on the work may be enhanced due to the same olfactory environment.

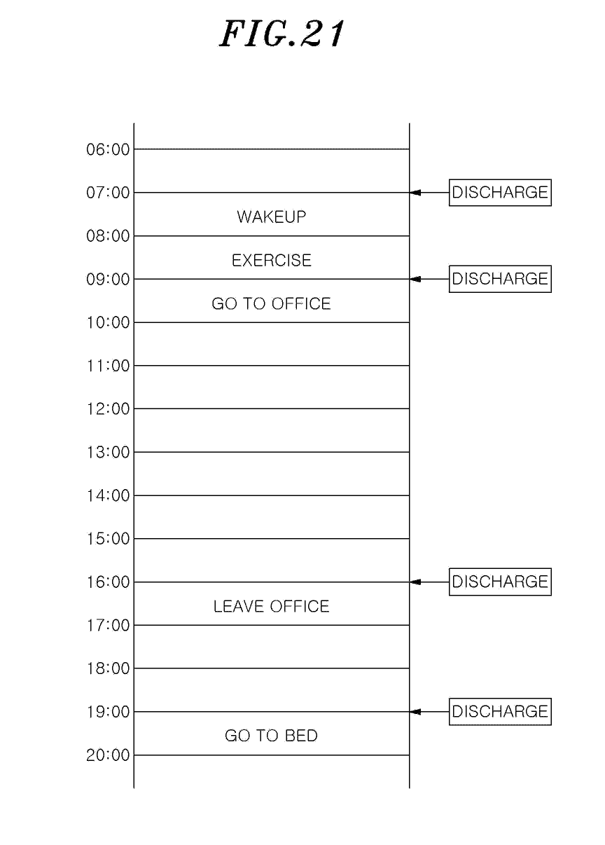

FIG. 21 illustrates an example in which a fragrance discharge device operates based on a user schedule according to an embodiment.

Referring to FIG. 21, a fragrance discharge device according to an embodiment may operate based on a schedule of a user. In detail, the user may input a daily schedule to the fragrance discharge device. The schedule may be directly input from the user through a UI of the fragrance discharge device and may be input through a smartphone of the user interacting with the fragrance discharge device. For example, the fragrance discharge device may determine a fragrance material that provides a pleasant and fresh feeling and may discharge the determined fragrance material before the user wakes up so that the user may have such pleasant and fresh feeling at wakeup. Also, when the user wakes up and then does some exercise, the fragrance discharge device may discharge a suitable fragrance material to remove an unpleasant feeling by sweat. Also, around a time at which the user leaves an office and comes home, the fragrance discharge device may determine and discharge a fragrance material suitable for rest so that the user may relax. Also, when the user is to take a sleep, the fragrance discharge device may determine and discharge a fragrance material suitable for the user to take a sound sleep.

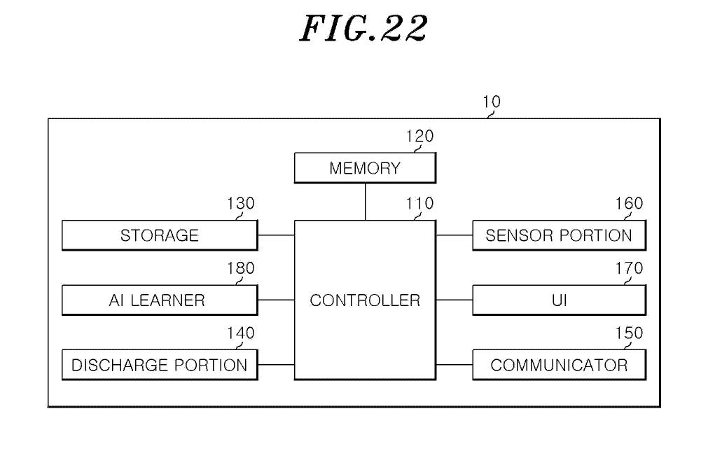

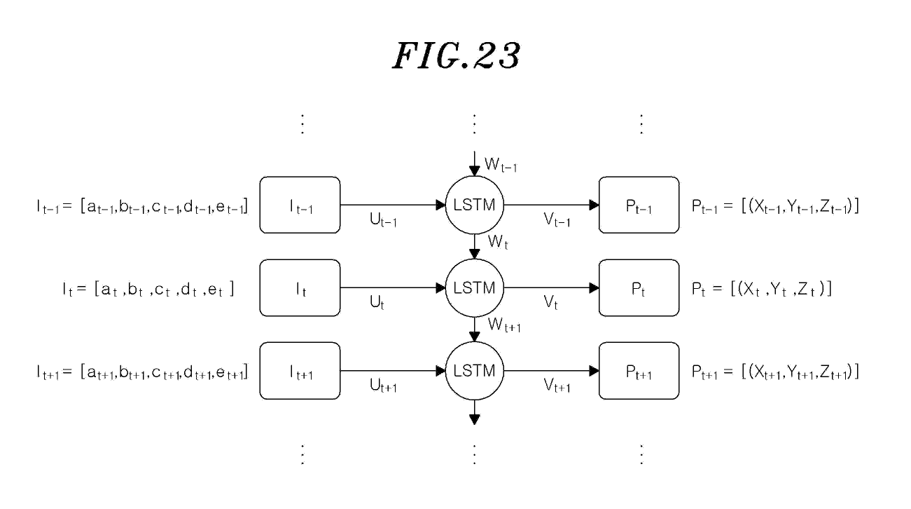

FIG. 22 is a block diagram illustrating a configuration of a fragrance discharge device according to an embodiment, and FIG. 23 illustrates a learning process of an artificial intelligence (AI) learner of FIG. 22.

Referring to FIGS. 22 and 23, the fragrance discharge device according to an embodiment may further include an AI learner 180 in addition to a configuration of the fragrance discharge device shown in FIGS. 1 and 2.

The controller 110 may control the discharge portion 140 to discharge a fragrance material based on context information including at least one of user information and environmental information around the fragrance discharge device 10.

The AI learner 180 may learn a control history of the controller 110 on discharging of a fragrance material based on the context information. The controller 110 may receive, from the AI learner 180, a recommendation on a control condition suitable for a situation in which the fragrance discharge device 10 operates to control discharging of the fragrance material. The AI learner 180 may use a control history from the past to the current time on discharging of the fragrance material with respect to the user information using a time series data analysis method, for example, a recurrent neural network (RNN). Here, the user information refers to information associated with a body or concept of the user and may include at least one of a location of the user in a space in which the fragrance discharge device 10 is installed, a relative location between the user and the fragrance discharge device 10, a type of a target toward which a fragrance material is to be discharged such as a person and an animal, olfactory sensitivity of the user, a preference of the user for a specific fragrance material, and body information such as emotion, stress, and concept of the user, a behavioral change or a movement of the user. The AI learner 180 may learn the control history on discharging of the fragrance material according to a change in the user information. The control history may be, for example, a type, a discharge amount, a discharge direction, and a discharge pattern of the fragrance material. The AI learner 180 may construct the control history as a database.

Also, the AI learner 180 may share the control history on discharging of the fragrance material with respect to a plurality of pieces of user information through a separate external server. The server may use the shared control history data for a time series data analysis.

Also, the controller 110 may control the discharge portion 140 using environmental information around the body 100 and the AI learner 180 may learn the control history of the controller 110 on discharging of the fragrance material by considering the environmental information. Here, the environmental information refers to information on an environment around the space in which the fragrance discharge device 10 is present, and may include at least one of characteristics of the space in which the fragrance discharge device 10 is present, for example, an ideal concept of space including a virtual space and a physical space such as a room, an office, a closet, a vehicle, and an airplane, illuminance, odor information, ventilation information, time information, and weather information.

The RNN may be suitable to learn a change in context over time in terms of AI learning. The RNN may analyze and learn an influence level of a primary factor based on state parameters of a previous time influencing over a current state. FIG. 23 illustrates the RNN. Here, an input vector (Iₜ) refers to context awareness based scent determination (CSD) information and may include at least one of presence or absence of the target, a location of the target, attribute information of the target, and the user information. Also, an output vector (Pₜ) refers to a CSD value and may be a type, discharge concentration, and a discharge interval of the fragrance material. Also, U, W, and V refer to parameter vectors and may be a recursive method of learning model by adjusting a weight of each parameter and using a result value acquired from the model as an input again. Each cell of the RNN may use a structure such as a long short-term memory (LSTM) and gated recurrent unit (GRU).

Referring to FIG. 23, I₀ if is input, P₁ that is an output value (CSD) in a subsequent time is predicted. The subsequent prediction may be associated with state information of a previous time. Accordingly, P₂ is predicted using an input of I₁ and by adding information of I₀. That is, an RNN learning model has an accumulated value of the previous time and applies context of the previous time. Learning of the RNN is performed to decrease a difference between a predicted value and an actual input value. Here, U, W, and V may be determined using a method of minimizing an error between a prediction value and an actual value of the learning model.

The aforementioned description is provided as an example only and thus, one of ordinary skill in the art to which the disclosure pertains may understand that various modifications may be made without departing from the technical spirit or features of the disclosure. Therefore, the aforementioned embodiments should be understood as examples and construed as not be limiting.

The scope of the disclosure is defined by the claims and the equivalents and all of the changes or modifications from the scope of the claims and the equivalents should be understood to be included in the scope of the disclosure.

What is claimed is:

1. A fragrance discharge device comprising:
a body onto which a storage is mounted, wherein the storage is configured to store fragrance material;
a discharge portion coupled to the body and configured to discharge, to an outside of the body, at least a portion of the fragrance material supplied from the storage; and
a controller comprising a processing device configured to recognize a presence or an absence of a target present around the body, and to control the discharge portion to discharge the fragrance material or to control a discharge direction of the fragrance material based on the presence or the absence of the target and a location of the target.

2. The fragrance discharge device of claim 1, wherein the controller is configured to control discharging of the fragrance material based on the presence or the absence of the target, the location of the target, and attribute information of the target, and the attribute information of the target comprises at least one of a size and a type of the target.

3. The fragrance discharge device of claim 1, further comprising:
a sensor portion coupled to the body and configured to detect the target present around the body.

4. The fragrance discharge device of claim 3, wherein the sensor portion comprises at least one of a location recognition sensor, a proximity sensor, a distance sensor, and an image sensor.

5. The fragrance discharge device of claim 1, wherein the controller is configured to adjust at least one of a type, an amount, a discharge direction, and a discharge pattern of the fragrance material that is discharged through the discharge portion.

6. The fragrance discharge device of claim 1, further comprising:
a communicator configured to communicably connect to an external device to control discharging of the fragrance material in conjunction with the external device.

7. The fragrance discharge device of claim 6, wherein the controller is configured to control discharging of the fragrance material based on the presence or the absence of the target, the location of the target, and attribute information of the target, and the target present around the body and the attribute information of the target are received from the external device.

8. The fragrance discharge device of claim 6, wherein the external device is a user device, and a user interface for providing a list of external devices interacting with the fragrance discharge device or controlling the fragrance discharge device is provided to the user device.

9. The fragrance discharge device of claim 6, wherein the controller is configured to control discharging of the fragrance material based on the presence or the absence of the target, the location of the target, and attribute information of the target,
the external device is a device capable of discharging the fragrance material, and
the controller is configured to control discharging of the fragrance material in conjunction with the external device based on the attribute information of the target.

10. The fragrance discharge device of claim 6, wherein the external device is a device capable of discharging the fragrance material, and
the controller is configured to control discharging of the fragrance material in conjunction with the external device based on a relative location between the external device and a user.

11. The fragrance discharge device of claim 6, wherein the external device is a multimedia player, and
the controller is configured to control discharging of the fragrance material based on content information of multimedia being played on the external device.

12. The fragrance discharge device of claim 2, wherein the controller is configured to control discharging of the fragrance material based on modified attribute information of the target in response to the attribute information of the target being modified.

13. The fragrance discharge device of claim 2, wherein the controller is configured to control the discharge portion based on user information of the fragrance discharge device with the presence or the absence of the target and the attribute information of the target.

14. The fragrance discharge device of claim 13, wherein the user information comprises at least one of a relative location between the fragrance discharge device and the user, body information of the user, and preference information of the user on a specific fragrance material.

15. The fragrance discharge device of claim 13, wherein the controller is configured to control the discharge portion using a scheme recommended by an Artificial Intelligence (AI) learner, AI learner configured to learn a control history of the controller on discharging of the fragrance material based on at least one of the presence or the absence of the target, the location of the target, and the attribute information of the target, and the user information, and to provide a result of the learning to the controller.

16. The fragrance discharge device of claim 15, wherein the AI learner is configured to learn the control history using a time series data analysis.

17. A fragrance discharge device comprising:
a discharge portion configured to connect to a storage and configured to discharge, to an outside of the body, fragrance material supplied from the storage, wherein the storage is configured to store the fragrance material;
a body onto which the storage is mounted;
a sensor portion configured to sense a presence and a location of a target present around the body;
a communicator configured to communicably connect to an external device; and
a controller comprising a processing device configured to control the discharge portion to discharge at least a portion of the fragrance material or to control a discharge direction of the fragrance material based on the presence or an absence of the target and the location of the target sensed by the sensor portion,
wherein the controller is configured to transmit at least one of a stored amount of the fragrance material and discharge information of the fragrance material to the external device through the communicator and to control the discharge portion based on a control signal received from the external device through the communicator.

18. The fragrance discharge device of claim 17, wherein the controller is configured to control discharging of the fragrance material based on the presence or the absence of the target, the location of the target, and attribute information of the target, and
at least a portion of the attribute information of the target is received from the external device.

19. A mountable fragrance container comprising:
a storage configured to store fragrance material, wherein:
the storage is configured to be mounted to a body, the body coupled to a discharge portion configured to discharge, to an outside of the body, at least a portion of the fragrance material supplied from the storage, and
the storage is further configured to be controlled by a controller, the controller comprising a processing device configured to recognize a presence or an absence of a target present around the body, and configured to control the discharge portion to discharge the fragrance material or to control a discharge direction of the fragrance material based on the presence or the absence of the target and a location of the target.
